(19) United States
(12) Patent Application Publication
(10) Pub. No.: US 2004/0204409 A1
(43) Pub. Date: Oct. 14, 2004

(54) Bicyclic compounds as NR2B receptor antagonists

(76) Inventors: Kazuo Ando, Aichi-ken (JP); Makoto Kawai, Aichi-ken (JP); Mitsuhiro Kawamura, Aichi-ken (JP); Miyako Matsumizu, Aichi-ken (JP); Asato Morita, Aichi-ken (JP); Isao Sakurada, Aichi-ken (JP)

Correspondence Address: WARNER-LAMBERT COMPANY, 2800 PLYMOUTH RD, ANN ARBOR, MI, 48105, US

(21) Appl. No.: 10/816,700

(22) Filed: Apr. 2, 2004

Related U.S. Application Data
(60) Provisional application No. 60/461,918, filed on Apr. 10, 2003.

(51) Int. Cl. A61K 31/537; A61K 31/495; C07D 265/30; C07D 211/26

(52) U.S. Cl. 514/235.2; 514/252.12; 514/317; 544/162; 544/399; 546/229

(57) ABSTRACT

This invention provides a compound of the formula (I):

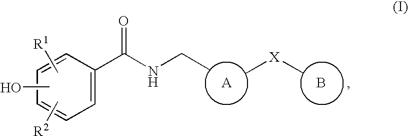

wherein R¹ and R² independently represent a hydrogen atom or the like; X represents a covalent bond or the like: A represents a bicyclic, aromatic, saturated or partially unsaturated heterocyclic or carbocyclic group having from 8 to 12 ring atoms; or the like: B represents a phenyl group or a heteroaryl group having from 5 to 6 ring atoms or the like:

These compounds are useful for the treatment of disease conditions caused by overactivation of NMDA NR2B receptor such of pain, or the like in mammalian. This invention also provides a pharmaceutical composition comprising the above compound.

This application is a United States utility application, which claims the benefit of priority to U.S. provisional application Serial No. 60/459,479 filed Apr. 10, 2003.

### TECHNICAL FIELD

This invention relates to novel bicyclic amide compounds. These compounds are useful as antagonists of NMDA (N-methyl-D-aspartate) NR2B receptor, and are thus useful for the treatment of pain, stroke, traumatic brain injury, Parkinson's disease, Alzheimer's disease, depression, anxiety, migraine, or the like in mammalian, especially humans. The present invention also relates to a pharmaceutical composition comprising the above compounds.

### BACKGROUND ART

Glutamate plays a dual role in the central nervous system (CNS) as essential amino acid and the principal excitatory neurotransmitters. There are two major classes of receptors, ionotoropic and metabotropic. Ionotropic receptors are classified into three major subclass, N-methyl-asparatate(NMDA), 2-amino-3(methyl-3-hydroxyisoxazol-4-yl)propionic acid (AMPA), kainate. There is considerable preclinical evidence that hyperalgesia and allodynia following peripheral tissue or nerve injury is not only due to an increase in the sensitivity of primary afferent nociceptors at the site of injury but also depends on NMDA receptor-mediated central changes in synaptic excitability. In humans, NMDA receptor antagonists have also been found to decrease both pain perception and sensitization. Also, overactivation of NMDA receptor is a key event for triggering neuronal cell death under pathological conditions of acute and chronic forms of neurodegeneration. However, while NMDA receptor inhibition has therapeutic utility in the treatment of pain and neurodegenerative diseases, there are significant liabilities to many available NMDA receptor antagonists that can cause potentially serious side effects. NMDA subunits are differentially distributed in the CNS. Especially, NR2B is believed to be restricted to the forebrain and laminas I and II of the dosal horn. The more discrete distribution of NR2B subunit in the CNS may support a reduced side-effect profile of agents that act selectively at this site.

For example, NMDA NR2B selective antagonists may have clinical utility for the treatment of neuropathic and other pain conditions in human with a reduced side-effect profile than existing NMDA antagonists (S. Boyce, et al., Neuropharmacology, 38, pp.611-623 (1999)).

WO 02/080928 discloses N-substituted nonaryl-heterocyclo amidyl compounds as NR2B antagonists.

### BRIEF DISCLOSURE OF THE INVENTION

It has now been found that bicyclic amide compounds are NMDA NR2B selective antagonists with analgesic activity by systemic administration. The compounds of the present invention may show less toxicity, good absorption, distribution, good solubility, low protein binding affinity, less drug-drug interaction, a reduced inhibitory activity at HERG channel and good metabolic stability.

The present invention provides a compound of the following formula (I):

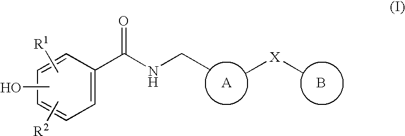

wherein

R¹ and R² independently represent a hydrogen atom, a halogen atom, an alkyl group having from 1 to 6 carbon atoms, an alkoxy group having from 1 to 6 carbon atoms, a cyano group, an alkanoyl group having from 1 to 6 carbon atoms, a haloalkyl group having from 1 to 6 carbon atoms, or a haloalkoxy group having from 1 to 6 carbon atoms;

X represents a covalent bond, an alkylene group having from 1 to 3 carbon atoms, an alkylene group having from 1 to 3 carbon atoms substituted by a hydroxy group or an oxo group; a methyleneoxy group, an ethyleneoxy group, a methyleneoxymethylene group, an oxymethylene group, an ethyleneoxy group, oxy, imino, iminomethylene, iminoethylene, methyleneimino or ethyleneimino,

said imino groups are unsubstituted or are substituted by an alkyl group having from 1 to 6 carbon atoms;

A represents a bicyclic, aromatic, saturated or partially unsaturated heterocyclic or carbocyclic group having from 8 to 12 ring atoms;

said heterocyclic group contains either from 1 to 4 nitrogen atoms, or 1 or 2 nitrogen atoms and/or 1 or 2 oxgen or sulfur atoms,

said heterocyclic or carbocyclic group are unsubstituted or are substituted by at least one substituent selected from the group consisting of substituents α;

B represents a phenyl group or a heteroaryl group having from 5 to 6 ring atoms; said phenyl groups and said heteroaryl groups having from 5 to 6 atoms are unsubstituted or are substituted by at least one substituent selected from the group consisting of substituents α;

said substituents α are selected from the group consisting of halogen atoms, alkyl groups having from 1 to 6 carbon atoms, alkoxy groups having from 1 to 6 carbon atoms, cyano groups, alkanoyl groups having from 1 to 6 carbon atoms, haloalkyl groups having from 1 to 6 carbon atoms, oxo groups or haloalkoxy groups having from 1 to 6 carbon atoms;

or a pharmaceutically acceptable ester of such compound;

or a pharmaceutically acceptable salt thereof.

The bicyclic amide compounds of this invention have an antagonistic action towards NMDA NR2B receptor subtype selectively and are thus useful in therapeutics, particularly for the treatment of stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse, alcohol abuse, Irritable bowel syndrome (IBS), or the like in mammalian, especially humans.

The compounds of the present invention are useful for the general treatment of pain, particularly neuropathic pain. Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57:1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD), Other types of pain include but are not limited to;

Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.

Central pain or ‘thalamic pain’ as defined by pain caused by lesion or dysfunction of the nervous system including but not limited to central post-stroke pain, multiple sclerosis, spinal cord injury, Parkinson's disease and epilepsy.

Heart and vascular pain including but not limited to angina, myocardical infarction, mitral stenosis, pericarditis, Raynaud's phenomenon, scleredoma, scleredoma, skeletal muscle ischemia.

Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.

Head pain including but not limited to migraine, migraine with aura, migraine without aura cluster headache, tension-type headache.

Orofacial pain including but not limited to dental pain, temporomandibular myofascial pain.

The present invention provides a pharmaceutical composition for the treatment of disease conditions caused by overactivation of NMDA NR2B receptor, in a mammalian subject, which comprises administering to said subject a therapeutically effective amount of a compound of formula (1).

Further, the present invention also provides a composition which comprises a therapeutically effective amount of the bicyclic amide compound of formula (I) or its pharmaceutically acceptable salt together with a pharmaceutically acceptable carrier. Among them, the composition is preferably for the treatment of disease defined above.

Also, the present invention provides for the use of a compound of formula (I), or a pharmaceutically acceptable ester of such compound, or a pharmaceutically acceptable salt thereof, as a medicament.

Also, the present invention provides a method for the treatment of disease conditions defined above, which comprises administering to said subject a therapeutically effective amount of a compound of formula (I).

Further, the present invention provides a method for the treatment of disease conditions defined above in a mammal, preferably human, which comprises administering to said subject a therapeutically effective amount of a compound of formula (I).

Yet further, the present invention provides the use of a therapeutically effective amount of a compound of formula (I) in the manufacture of a medicament for the treatment of the disease conditions defined above.

### DETAILED DESCRIPTION OF THE INVENTION

As used herein, the term “halogen” means fluoro, chloro, bromo and iodo, preferably fluoro or chloro.

As used herein, the term “alkyl” means straight or branched chain saturated radicals, including, but not limited to methyl, ethyl, n-propyl, isopropyl, n-butyl, iso-butyl, secondary-butyl, tertiary-butyl.

As used herein, the term “alkoxy” means alkyl-O—, including, but not limited to methoxy, ethoxy, n-propoxy, isopropoxy, n-butoxy, iso-butoxy, secondary-butoxy, tertiary-butoxy.

As used herein, the term “inino” means —NH—.

As used herein, the term “alkanoyl” means a group having carbonyl such as R′—C(O)— wherein R′ is H, C₁₋₅ alkyl, phenyl or C₃₋₆ cycloalkyl, including, but not limited to formyl, acetyl, ethyl-C(O)—, n-propyl-C(O)—, isopropyl-C(O)—, n-butyl-C(O)—, iso-butyl-C(O)—, secondary-butyl-C(O)—, tertiary-butyl-C(O)—, cyclopropyl-C(O)—, cyclobutyl-C(O)—, cyclopentyl-C(O)—, cyclohexyl-C(O)—, and the like.

As used herein, the term “aryl” means a monocyclic aromatic carbocyclic ring of 5 to 10 carbon atoms, including, but not limited to, phenyl or naphthyl.

The term “heteroaryl” means a 5- to 6-membered aromatic hetero mono-cyclic ring which consists of from 1 to 4 heteroatoms independently selected from the group consisting of sulfur atoms, oxygen atoms and nitrogen atoms including, but not limited to, pyrazolyl, furyl, thienyl, oxazolyl, tetrazolyl, thiazolyl, imidazolyl, thiadiazolyl, pyridyl, pyrimidinyl, pyrrolyl, thiophenyl, pyrazinyl, pyridazinyl, isooxazolyl, isothiazolyl, triazolyl, furazanyl, and the like.

The term “alkylene”, as used herein, means a saturated hydrocarbon (straight chain or branched) wherein a hydrogen atom is removed from each of the terminal carbons such as methylene, ethylene, methylethylene, propylene, butylene, pentylene, hexylene and the like.

The term “bicyclic, aromatic, saturated or partially unsaturated heterocyclic group”, as used herein, means a 8 to 12-membered bicyclic, aromatic, saturated or partially unsaturated ring, which contains either from 1 to 4 nitrogen atoms, or 1 or 2 nitrogen atoms and/or 1 or 2 oxgen or sulfur atoms; and wherein a hydrogen atom is removed from each of the terminal carbons. Examples of such groups include, but are not limited to, tetrahydroquinoline, tetrahydroisoquinoline, decahydroquinoline, octahydroisoquinoline, benzimidazole, indole, isoindole, indoline, isoindoline, benzothiophene, benzofurane, indolizine, indazole, benzoxazole, benzthiazole, chroman, isochroman, quinoline, isoquinoline, quinoxaline or quinazoline.

The term “bicyclic, aromatic, saturated or partially unsaturated carbocyclic group”, as used herein, means a 8 to 12-membered bicyclic, aromatic, saturated or partially unsaturated ring; and wherein a hydrogen atom is removed from each of the terminal carbons. Examples of such groups include, but are not limited to, naphthalene, indan, indene, 1,2,3,4-tetrahydronaphthalene, bicyclo[3.3.0]octylene, bicyclo[3.2.1 ]octylene or bicyclo[3.3.1 ]nonylene.

The term “haloalkyl”, as used herein, means an alkyl radical which is substituted by halogen atoms as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl groups and the like.

The term “haloalkoxy”, as used herein, means haloalkyl-O—, including, but not limited to, fluoromethoxy, difluoromethoxy, trifluoromethoxy, 2-fluoroethoxy, 2,2-difluoroethoxy, 2,2,2-trifluoroethoxy, 2,2,2-trichloroethoxy, 3-fluoropropoxy, 4-fluorobutoxy, chloromethoxy, trichloromethoxy, iodomethoxy and bromomethoxy groups and the like.

Where the compounds of formula (I) contain hydroxy groups, they may form esters. Examples of such esters include esters with a hydroxy group and esters with a carboxy group. The ester residue may be an ordinary protecting group or a protecting group which can be cleaved in vivo by a biological method such as hydrolysis.

The term “ordinary protecting group” means a protecting group, which can be cleaved by a chemical method such as hydrogenolysis, hydrolysis, electrolysis or photolysis.

The term “esters” means a protecting group which can be cleaved in vivo by a biological method such as hydrolysis and forms a free acid or salt thereof. Whether a compound is such a derivative or not can be determined by administering it by intravenous injection to an experimental animal, such as a rat or mouse, and then studying the body fluids of the animal to determine whether or not the compound or a pharmaceutically acceptable salt thereof can be detected.

Preferred examples of groups for an ester of a hydroxy group include: lower aliphatic alkanoyl groups, for example: alkanoyl groups, such as the formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl and henicosanoyl groups; halogenated alkylcarbonyl groups, such as the chloroacetyl, dichloroacetyl, trichloroacetyl, and trifluoroacetyl groups; alkoxyalkylcarbonyl groups, such as the methoxyacetyl group; and unsaturated alkylcarbonyl groups, such as the acryloyl, propioloyl, methacryloyl, crotonoyl, isocrotonoyl and (E)-2-methyl-2-butenoyl groups; more preferably, the lower aliphatic alkanoyl groups having from 1 to 6 carbon atoms; aromatic alkanoyl groups, for example: arylcarbonyl groups, such as the benzoyl, α-naphthoyl and β-naphthoyl groups; halogenated arylcarbonyl groups, such as the 2-bromobenzoyl and 4-chlorobenzoyol groups; lower alkylated arylcarbonyl groups, such as the 2,4,6-trimethylbenzoyl and 4-toluoyl groups; lower alkoxylated arylcarbonyl groups, such as the 4-anisoyl group; nitrated arylcarbonyl groups, such as the 4-nitrobenzoyl and 2-nitrobenzoyl groups; lower alkoxycarbonylated arylcarbonyl groups, such as the 2-(methoxycarbonyl)benzoyl group; and arylated arylcarbonyl groups, such as the 4-phenylbenzoyl group; alkoxycarbonyl groups, for example: lower alkoxycarbonyl groups, such as the methoxycarbonyl, ethoxycarbonyl, propoxycarbonyl, butoxycarbonyl, sec-butoxycarbonyl, t-butoxycarbonyl and isobutoxycarbonyl groups; and halogen- or tri(lower alkyl)silyl-substituted lower alkoxycarbonyl groups, such as the 2,2,2-trichloroethoxycarbonyl and 2-trimethylsilylethoxycarbonyl groups; tetrahydropyranyl or tetrahydrothiopyranyl groups, such as: tetrahydropyran-2-yl, 3-bromotetrahydropyran-2-yl, 4-methoxytetrahydropyran-4-yl, tetrahydrothiopyran-2-yl, and 4-methoxytetrahydrothiopyran-4-yl groups; tetrahydrofuranyl or tetrahydrothiofuranyl groups, such as: tetrahydrofuran-2-yl and tetrahydrothiofuran-2-yl groups; silyl groups, for example: tri(lower alkyl)silyl groups, such as the trimethylsilyl, triethylsilyl, isopropyldimethylsilyl, t-butyldimethylsilyl, methyldiisopropylsilyl, methyldi-t-butylsilyl and triisopropylsilyl groups; and tri(lower alkyl)silyl groups substituted by 1 or 2 aryl groups, such as the diphenylmethylsilyl, diphenylbutylsilyl, diphenylisopropylsilyl and phenyldiisopropylsilyl groups; alkoxymethyl groups, for example: lower alkoxymethyl groups, such as the methoxymethyl, 1,1-dimethyl-1-methoxymethyl, ethoxymethyl, propoxymethyl, isopropoxymethyl, butoxymethyl and t-butoxymethyl groups; lower alkoxylated lower alkoxymethyl groups, such as the 2-methoxyethoxymethyl group; and halo(lower alkoxy)methyl groups, such as the 2,2,2-trichloroethoxymethyl and bis(2-chloroethoxy)methyl groups; substituted ethyl groups, for example: lower alkoxylated ethyl groups, such as the 1-ethoxyethyl and 1-(isopropoxy)ethyl groups; and halogenated ethyl groups, such as the 2,2,2-trichloroethyl group; aralkyl groups, for example: lower alkyl groups substituted by from 1 to 3 aryl groups, such as the benzyl, α-naphthylmethyl, β-naphthylmethyl, diphenylmethyl, triphenylmethyl, α-naphthyldiphenylmethyl and 9-anthrylmethyl groups; and lower alkyl groups substituted by from 1 to 3 substituted aryl groups, where one or more of the aryl groups is substituted by one or more lower alkyl, lower alkoxy, nitro, halogen or cyano substituents, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; alkenyloxycarbonyl groups: such as the vinyloxycarbonyl and aryloxycarbonyl groups; and aralkyloxycarbonyl groups in which the aryl ring may be substituted by 1 or 2 lower alkoxy or nitro groups: such as the benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl groups.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

According to formula (I), the hydroxy phenyl group is preferably para-hydroxy phenyl.

A preferred compound of formula (I) of this invention is that wherein R¹ and R² independently represent a hydrogen atom, a halogen atom or an alkyl group having from 1 to 6 carbon atoms, more preferably a hydrogen atom, a fluorine atom, a chlorine atom or an alkyl group having from 1 to 3 carbon atoms. Most preferably R¹ and R² independently represent a hydrogen atom or a fluorine atom.

A preferred compound of formula (I) of this invention is that wherein X represents an alkylene group having from 1 to 2 carbon atoms, an alkylene group having from 1 to 2 carbon atoms substituted by a hydroxy group or an oxo group, a methyleneoxy group, an oxymethylene group, iminomethylene or methyleneimino, said imino groups are unsubstituted or are substituted by an alkyl group having from 1 to 6 carbon atoms. More preferably, X represents an alkylene group having from 1 to 2 carbon atoms, an alkylene group having from 1 to 2 carbon atoms substituted by a hydroxy group, an oxymethylene group or iminomethylene. Most preferably, X represents an alkylene group having from 1 to 2 carbon atoms, an oxymethylene group or iminomethylene.

A suitable compound of formula (I) of this invention is that wherein A represents an optionally substituted bicyclic aromatic, saturated or partially unsaturated heterocyclic group having from 8 to 12 ring atoms, said heterocyclic group contains either from 1 to 3 nitrogen atoms, or 1 nitrogen atom and/or 1 oxygen or sulfur atom. Preferably, A represents a bicyclic aromatic heterocyclic group having from 8 to 10 ring atoms, said heterocyclic group contains either from 1 to 3 nitrogen atoms, or 1 nitrogen atom and/or 1 oxygen atom. More preferably, A represents a benzimidazole group, a benzoisoxazole group, an indole group, an indazole group, a quinazoline group, an oxo-1H-benzimidazole group, an imidazopyridine group, a tetrahydroimidazopyridine group, a quinoline group, a benzoxazole group, a benzthiazole group or a quinoxaline group. Most preferably, A represents a benzimidazole group, a benzoisoxazole group, an indole group, an indazole group, a quinazolin group, an oxo-1H-benzimidazole group, an imidazopyridine group, a tetrahydroimidazopyridine group, or a quinoline group. Where A is substituted, A is suitably substituted by alkyl having 1 to 6 carbons e.g. methyl.

A preferred compound of formula (I) of this invention is that wherein B represents an optionally substituted phenyl group, more preferably unsubstituted phenyl or a fluorophenyl group

Particularly preferred compounds of the invention include those in which each variable in Formula (I) is selected from the preferred groups for each variable. Even more preferable compounds of the invention include those where each variable in Formula (I) is selected from the more preferred or most preferred groups for each variable.

A preferred individual compound of this invention is selected from

N-[(2-benzyl-1H-benzimidazol-5-yl)methyl]-4-hydroxybenzamide;

4-hydroxy-N-{[1-(2-phenylethyl)-1H-benzimidazol-6-yl]methyl}benzamide;

N-[(2-benzyl-1H-indol-5-yl)methyl]-4-hydroxybenzamide;

4-hydroxy-N-{[1-(2-phenylethyl)-1H-indazol-6-yl]methyl}benzamide;

N-{[4-(Benzylamino)quinazolin-6-yl]methyl}-4-hydroxybenzamide;

4-hydroxy-N-{[2-methyl-1-(2-phenylethyl)-1H-benzimidazol-6-yl]methyl}benzamide;

N-{[4-(Benzyloxy)quinolin-6-yl]methyl}-4-hydroxybenzamide; 4-hydroxy-N-{[2-oxo-3-(2-phenylethyl)-2,3-dihydro-1H-benzimidazol-5-yl]methyl}benzamide;

4-hydroxy-N-{[3-(2-phenylethyl)-1H-indazol-5-yl]methyl}benzamide];

4-Hydroxy-N-{[3-(2-phenylethyl)imidazo[1,5-a]pyridin-6-yl]methyl}benzamide;

N-{[3-(benzyloxy)-1,2-benzisoxazol-5-yl]methyl}-4-hydroxybenzamide;

N-{[2-(2-fluorobenzyl)-1H-benzimidazol-6-yl]methyl}-4-hydroxybenzamide;

N-[(2-benzyl-5,6,7,8-tetrahydroimidazo[1,2-a]pyridin-7-yl)methyl]-4-hydroxybenzamide;

N-[(2-benzyl-1H-indol-5-yl)methyl]-3-fluoro-4-hydroxybenzamide; and

4-hydroxy-N-{[1-(2-phenylethyl)-1H-imidazo[4,5-b]pyridin-6-yl]methyl}benzamide;

or a pharmaceutically acceptable salt thereof.

A further preferred individual compound of this invention is selected from

4-hydroxy-N-{[1-(2-phenylethyl)-1H-benzimidazol-6-yl]methyl}benzamide;

N-[(2-benzyl-1H-indol-5-yl)methyl]-4-hydroxybenzamide;

4-hydroxy-N-{[1-(2-phenylethyl)-1H-indazol-6-yl]methyl}benzamide;

N-{[4-(Benzylamino)quinazolin-6-yl]methyl}-4-hydroxybenzamide;

4-hydroxy-N-{[2-methyl-1-(2-phenylethyl)-1H-benzimidazol-6-yl]methyl}benzamide;

4-hydroxy-N-{[2-oxo-3-(2-phenylethyl)-2,3-dihydro-1H-benzimidazol-5-yl]methyl}benzamide;

4-hydroxy-N-{[3-(2-phenylethyl)-1H-indazol-5-yl]methyl}benzamide];

4-Hydroxy-N-{[3-(2-phenylethyl)imidazo[1,5-a]pyridin-6-yl]methyl}benzamide;

N-{[3-(benzyloxy)-1,2-benzisoxazol-5-yl]methyl}-4-hydroxybenzamide;

N-[(2-benzyl-5,6,7,8-tetrahydroimidazo[1,2-a]pyridin-7-yl)methyl]-4-hydroxybenzamide;

N-[(2-benzyl-1H-indol-5-yl)methyl]-3-fluoro-4-hydroxybenzamide; and

4-hydroxy-N-{[1-(2-phenylethyl)-1H-imidazo[4,5-b]pyridin-6-yl]methyl}benzamide;

or a pharmaceutically acceptable salt thereof.

### General Synthesis

The compounds of the present invention may be prepared by a variety of processes well known for the preparation of compounds of this type, for example as shown in the following reaction Schemes. Unless otherwise indicated R¹, R², A, B, and X in the reaction Schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1991);

The following reaction Schemes illustrate the preparation of compounds of formula (I).

Scheme 1:

This illustrates the preparation of compounds of formula (I).

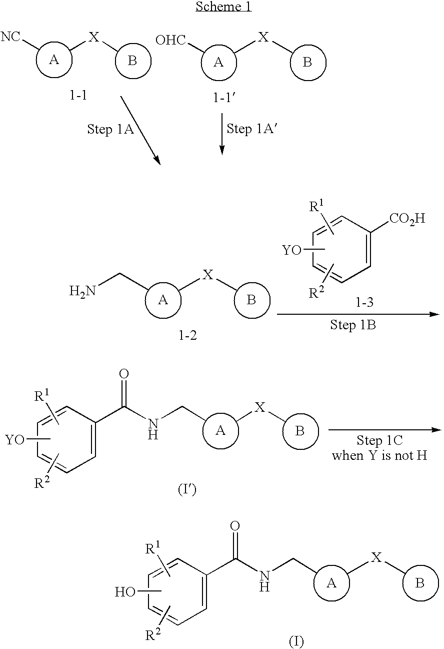

In the above formula, Y represents a hydrogen atom or a protecting group.

Step 1A

In this Step, an amine compound of formula 1-2 can be prepared by the reduction of a cyano compound of formula 1-1 under known hydrogenation conditions in the presence of a metal catalyst, e.g. Raney nickel catalysts, palladium catalysts or platinum catalysts, preferably Raney nickel catalysts in an inert solvent, e.g. acetic acid, alcohols, such as methanol, ethanol; ethyl acetate, tetrahydrofuran, and N,N-dimethylformamide. If desired, this reaction may be carried out in the presence or absence of an additive such as ammonium hydroxide.

Step 1A′

In this Step, the amine compound of formula 1-2 also can be prepared from an aldehyde compound of formula 1-1′.

The aldehyde compound of formula 1-1′ may be first subjected to oxime formation treating with hydroxylamine acid salt, such as hydroxylamine hydrochloride, in a suitable solvent, such as an alcohol, such as methanol or ethanol, optionally in the presence of a base, such as an alkaline earth metal hydroxide, carbonate, such as sodium hydroxide, potassium hydroxide, sodium carbonate or potassium carbonate, followed by reduction in the presence of a suitable reducing agent in a reaction inert solvent, such as LiAlH₄, LiBH₄, Fe, Sn or Zn in a suitable solvent, e.g. an acid, such as acetic acid, to afford a corresponding the amine compound of formula 1-2.

Step 1B

In this Step, an amide compound of formula (I′) can be prepared by the coupling reaction of an amine compound of formula 1-2 with an acid compound of formula 1-3 in the presence or absence of a coupling reagent, e.g. diimides (e.g., 1-(3-dimethylaminopropyl)-3-ethylcarbodiimide hydrochloride(EDCI), dicyclohexylcarbodiimide (DCC), water soluble carbodiimide (WSC)), 2-ethoxy-N-ethoxycarbonyl-1,2-dihydroquinoline, benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, or ethyl chloroformate, in an inert solvent, e.g. acetone, dimethylformamide, acetonitrile; halogenated hydrocarbons, such as dichloromethane, dichloroethane, chloroform; and ethers, such as tetrahydrofuran and dioxane. If desired, this reaction may be carried out in the presence of an additive such as 1-hydoroxybenzotriazole or 1-hydroxyazabenzotriazole or in the presence of a base such as N-methylmorpholine.

Step 1C

When Y is not a hydrogen atom, In this Step, a compound of formula (I) may be prepared by the deprotection of the compound of formula (I′), according to known procedures such as those described in Protective Groups in Organic Synthesis edited by T. W. Greene et al. (John Wiley & Sons, 1991).

Scheme 2:

This illustrates the alternative preparation of the intermediate compound of formula 1-2.

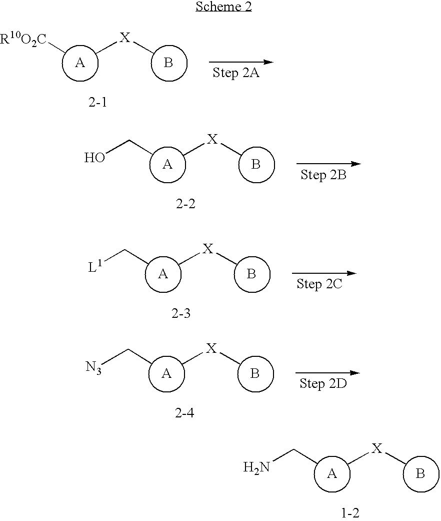

In the above formula, R¹⁰ represents a hydrogen atom or an alkyl group having from 1 to 6 carbon atoms. L¹ represents a leaving group. Example of suitable leaving groups include: halogen atoms, such as chlorine, bromine and iodine; sulfonic esters such as TfO (triflates), MsO (mesylates), TsO (tosylates); and the like.

Step 2A

In this step, a compound of formula 2-1 may be subjected to reduction to give an alcohol compound of formula 2-2. The reduction may be carried out in the presence of a suitable reducing agent e.g. LiAlH₄, diisobutylalminum hydride(DIBAL-H) or LiBH₄ in a reaction inert solvent, e.g. aliphatic hydrocarbons, such as hexane, heptane and petroleum ether; aromatic hydrocarbons, such as benzene, toluene, o-dichlorobenzene, and xylene; ethers, such as diethyl ether, diisopropyl ether, tetrahydrofuran(THF), diglyme and dioxane, preferably the ethers.

Step 2B

In this Step, the alcohol compound of formula 2-2, prepared as described in Step 2A may be converted to compound with a leaving group L¹ of formula 2-3 under conditions known to those skilled in the art.

For example, the hydroxy group of the compound of formula 2-2 may be converted to the halogen atom using a halogenating agent, e.g. thionyl chloride, oxalyl chloride, para-toluenesulfonyl chloride, methanesulfonyl chloride, hydrogen chloride, phosphorus trichloride, phosphorus pentachloride, N-chlorosuccinimide (NCS), phosphorus oxychloride, trimethylsilyl chloride or phosphorus reagents such as triphenylphosphine, tributyl phosphine or triphenylphosphite in the presence of halogen source such as carbon tetrachloride, chlorine, NCS; brominating agents, such as hydrogen bromide, N-bromosuccinimide (NBS), phosphorus tribromide, trimethylsilyl bromide or phosphorus reagents such as triphenylphosphine, tributyl phosphine or triphenylphosphite in the presence of halogen source such as carbon tetrabromide, bromine or NBS; and iodinating agents, such as hydroiodic acid, phosphorus triiodide, or phosphorus reagents such as triphenylphosphine, tributyl phosphine or triphenylphosphite in the presence of halogen source such as iodine, in the presence or absence of a reaction inert solvent, e.g. aliphatic hydrocarbons, such as hexane, heptane and petroleum ether; aromatic hydrocarbons, such as benzene, toluene, o-dichlorobenzene, nitrobenzene, pyridine, and xylene; halogenated hydrocarbons, such as methylene chloride, chloroform, carbon tetrachloride and 1,2-dichloroethane; and ethers, such as diethyl ether, diisopropyl ether, tetrahydrofuran and dioxane, preferably the aromatic hydrocarbons, halogenated hydrocarbons and ethers.

Alternatively, a hydroxy group of the compound of formula 1-2a may be converted to the sulfonate group using a sulfonating agent, e.g. para-toluenesulfonyl chloride, para-toluenesulfonic anhydride, methanesulfonyl chloride, methanesulfonic anhydride, trifluoromethanesulfonic anhydride in the presence of, or absence of a base, e.g. an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride, or an amine such as triethylamine, tributylamine, diisopropylethylamine, pyridine or dimethylaminopyridine in the presence or absence of a reaction inert solvent, e.g. aliphatic hydrocarbons, such as hexane, heptane and petroleum ether; aromatic hydrocarbons, such as benzene, toluene, o-dichlorobenzene, nitrobenzene, pyridine, and xylene; halogenated hydrocarbons, such as methylene chloride, chloroform, carbon tetrachloride and 1,2-dichloroethane; and ethers, such as diethyl ether, diisopropyl ether, tetrahydrofuran and dioxane; N,N-dimethylformamide, and dimethylsulfoxide

Step 2C

In this Step, an azide compound of formula 2-4 may be prepared by the nucleophilic displacement of the above obtained compound of formula 2-3 with azide agents, e.g. sodium azide or lithium azide, in an inert solvent, e.g. water; aromatic hydrocarbons, such as benzene, toluene, o-dichlorobenzene, nitrobenzene, pyridine, and xylene; ethers, such as tetrahydrofuran and dioxane. N,N-dimethylformamide, and dimethoxyethane. Of these solvents, we prefer the water and N,N-dimethylformamide. This reaction may be carried out in the presence of a suitable additive agent, e.g. sodium iodide, potassium iodide, 1,4,7,10.13-pentaoxacyclopentadecane(15-Crown-5) or 1,4,7,10-tetraoxacyclododecane(12-Crown-4).

Step 2D

In this Step, the amine compound of formula 1-2 may be prepared by carrying out reduction of the azide compound of formula 2-4, prepared as described in Step 2C.

The reduction may also be carried out under known hydrogenation conditions in the presence of a metal catalyst such as Lindlar catalysts, Raney nickel catalysts, palladium catalysts or platinum catalysts (preferably Lindlar catalysts, palladium catalysts or platinum catalysts). This reaction may be carried out under hydrogen atmosphere in a reaction inert solvent, e.g. acetic acid, alcohols, such as methanol, ethanol; ethyl acetate, tetrahydrofuran, and N,N-dimethylformamide, preferably the alcohols.

Scheme 3:

This illustrates a preparation of an intermediate compound of formula 3-5, which corresponds to the intermediate compound of formula 1-1 wherein the ring A portion contains azole moiety.

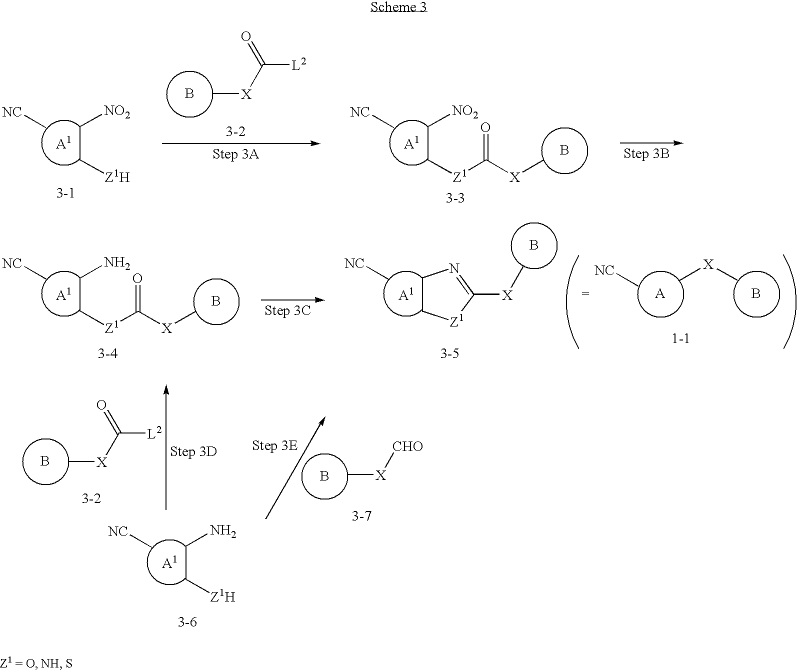

In the above formula, A¹ represents a monocyclic, aromatic, saturated or partially unsaturated heterocyclic or carbocyclic group having from 5 to 9 ring atoms; said heterocyclic group contains either from 1 to 2 nitrogen atoms, or 1 or 2 oxgen or sulfur atoms; said heterocyclic or carbocyclic group are unsubstituted or are substituted by at least one substituent selected from the group consisting of substituents α; said substituents α are selected from the group consisting of halogen atoms, alkyl groups having from 1 to 6 carbon atoms, alkoxy groups having from 1 to 6 carbon atoms, cyano groups, alkanoyl groups having from 1 to 6 carbon atoms, haloalkyl groups having from 1 to 6 carbon atoms, oxo groups or haloalkoxy groups having from 1 to 6 carbon atoms;

Examples of said heterocyclic or carbocyclic group include, but are not limited to, cyclopentane, cyclopentene, cyclohexane, cyclohexene, phenyl, cycloheptane, cycloheptene, pyrrole, thiophene, furan, imidazole, pyrazole, thiazole, oxazole, pyridine, pyrazine, pyrimidine, pyridazine, piperidine, piperazine or morpholine L² represents a halogen atom such as, chlorine, bromine or iodine.

Z¹ represents O, NH or S.

Step 3A

In this Step, an amide compound of formula 3-3 may be prepared by acylation of an amine compound of formula 3-1 with acylating agents, e.g. an acid halide, an acid anhydride or trialkyl orthoformate, in an inert solvent, e.g. aromatic hydrocarbons, such as benzene, toluene and xylene; halogenated hydrocarbons, such as methylene chloride, chloroform, carbon tetrachloride and dichloroethane; ethers, such as diethyl ether, diisopropyl ether, tetrahydrofuran and dioxane; and pyridine. This reaction may be carried out in the presence or absence of a base, e.g. pyridine, picoline, 4-(N,N-dimethylamino)pyridine, triethylamine, tributylamine, diisopropylethylamine, N-methylmorphorine and N-methylpiperidine.

Step 3B

In this Step, a diamino compound of formula 3-4 may be prepared by the reduction of an nitro compound of formula 3-3, prepared as described in Step 3A with a reducing agent in an inert solvent, e.g. methanol, ethanol, ethyl acetate, THF or mixtures thereof. The reduction may be carried out under known hydrogenation conditions in the presence of a metal catalyst, e.g. nickel catalysts such as Raney nickel, palladium catalysts such as Pd—C, platinum catalysts such as PtO₂, or ruthenium catalysts such as RuCl₂ (Ph₃P)₃ under hydrogen atmosphere or in the presence of hydrogen sources such as hydrazine or formic acid. If desired, the reaction is carried out under acidic conditions, e.g. in the presence of hydrochloric acid or acetic acid. The reduction may also be carried out in the presence of a suitable reducing agent, e.g. LiAlH₄, LiBH₄, Fe, Sn or Zn, in a reaction inert solvent, e.g. methanol, ethanol, diglyme, benzene, toluene, xylene, o-dichlorobenzene, dichloromethane, dichloroethane, tetrahydrofuran, dioxane, or mixtures thereof; or without solvent. If desired, when a reducing reagent is Fe, Sn or Zn, the reaction is carried out under acidic conditions in the presence of water.

Step 3C

In this Step, an azole compound of formula 3-5 may be prepared by the cyclization of the diamino compound of formula 3-4, prepared as described in Step 3B under conditions known to those skilled in the art. The compound of formula 3-4 may be cyclized to form an azole ring by any synthetic procedure applicable to structure-related compounds known to those skilled in the art (for example, see Milata Liktor et al., Heterocycles, 2001, 55(5), 905-924,). For example, this reaction may be carried out in a reaction inert solvent, e.g. benzene, toluene, xylene, o-dichlorobenzene, nitrobenzene, dichloromethane, dichloroethane, tetrahydrofuran (THF), dimethylformamide (DMF), dioxane, dimethylsulfoxide (DMSO) or mixtures thereof, in the presence or absence of a catalyst such as para-toluenesulfonic acid, camphorsulfonic acid, acetic acid or trifluoroacetic acid.

Step 3D

In this Step, the diamino compound of formula 3-4 may be prepared by acylation of the compound of formula 3-6. This reaction is essentially the same as and may be carried out in the same manner as and using the same reagents and reaction conditions as Step 3A in Scheme 3.

Step 3E

In this Step, the azole compound of formula 3-5 may be prepared by the cyclization of the diamino compound of formula 3-4 with an aldehyde compound of formula 3-7. The reaction may be normally and preferably effected in the presence of a solvent, e.g. aromatic hydrocarbons, such as benzene, toluene, xylene and nitrobenzene; alcohols, such as methanol and ethanol.

Scheme 4:

This illustrates a preparation of intermediate compounds of formula 4-4 and 4-7.

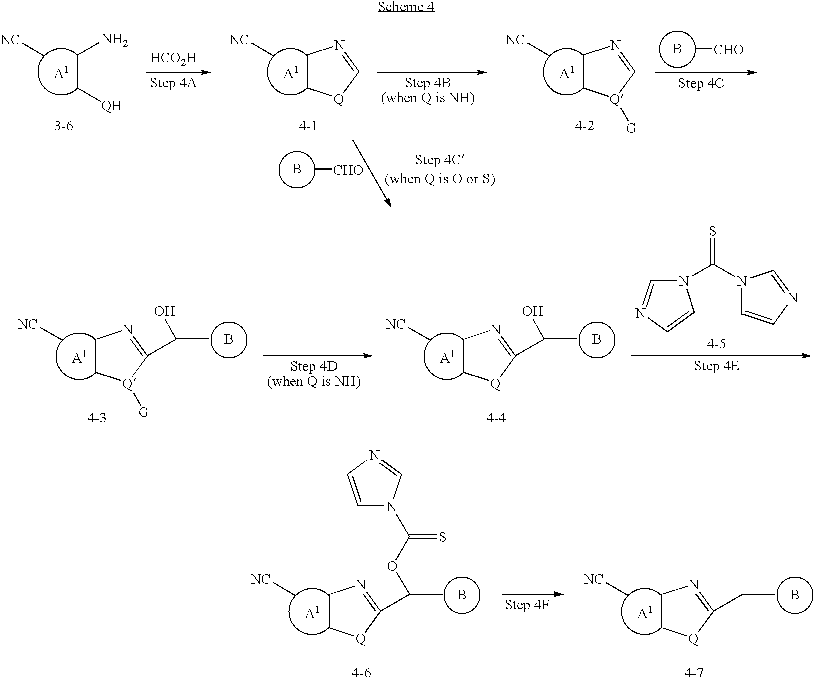

In the above formula, A¹ is defined in Scheme 3. Q represents O, NH or S. Q′ represents N. G represents a protecting group.

Step 4A

In this Step, an azole compound of formula 4-1 may be prepared by the cyclization of the diamino compound of formula 3-6 with formic acid. The reaction may be carried out in the presence or absence of a solvent, e.g. formic acid itself, H₂O, or aromatic hydrocarbons, such as benzene, toluene and xylene.

Step 4B

In these Steps, a protected compound of formula 4-2 wherein Q′ is N may be prepared from a compound of formula 4-1 by converting the NH group into a protected N group. The step may be carried out by using, for example, the compound of formula 4-1, appropriate triethyl orthoformate, silyl halides, aralkyl halide, acid halides, acid anhydride and acids, such as benzyl, t-butyldimethylsilyl (TBS) chloride, t-butyldiphenylsilylchloride, Z-chloride and t-BocCl or Boc₂O, using the methods described in Protective Groups in Organic Synthesis edited by T. W. Greene et al. (John Wiley & Sons, 1991). Of these reagents, we prefer triethyl orthoformate. The reaction may be carried out in the presence or absence of a solvent, e.g. aromatic hydrocarbons, such as benzene, toluene and xylene; halogenated hydrocarbons, such as methylene chloride, chloroform, carbon tetrachloride and dichloroethane; and ethers, such as diethyl ether, diisopropyl ether, tetrahydrofuran and dioxane; and DMF and DMSO. This reaction may be carried out in the presence or absence of a catalyst, e.g. para-toluenesulfonic acid, camphorsulfonic acid, and acetic acid.

Step 4C

In this Step, a 2-substituted azole compound of formula 4-3, wherein Q′ is N, may be prepared by the reaction of the compound of formula 4-2 wherein Q′ in N, with an aldehyde compound in an inert solvent, e.g. aliphatic hydrocarbons, such as hexane, heptane and petroleum ether; aromatic hydrocarbons, such as benzene, toluene o-dichlorobenzene, nitrobenzene, and xylene; and ethers, such as diethyl ether, diisopropyl ether, tetrahydrofuran and dioxane. This reaction may be carried out in the presence of a base, e.g. lithium, alkyllithium, such as n-butyllithium, tert-butyllithiun, sec-butyllithium, aryllithium such as phenylithium.

Step 4C′

In this Step, a 2-substituted azole compound of formula 4-4, wherein Q is O or S, may be prepared by the reaction of the compound of formula 4-1 with an aldehyde compound in an inert solvent, e.g. aliphatic hydrocarbons, such as hexane, heptane and petroleum ether; aromatic hydrocarbons, such as benzene, toluene o-dichlorobenzene, nitrobenzene, and xylene; and ethers, such as diethyl ether, diisopropyl ether, tetrahydrofuran and dioxane. This reaction may be carried out in the presence of a base, e.g. lithium, alkyllithium, such as n-butyllithium, tert-butyllithiun, sec-butyllithium, aryllithium such as phenylithium.

Step 4D

In this Step, a 2-substituted azole compound of formula 4-4, wherein Q is NH, may be prepared by the deprotection of the compound of formula 4-3 wherein Q′ is N, prepared as described in Step 4C, according to known procedures such as those described in Protective Groups in Organic Synthesis edited by T. W. Greene et al. (John Wiley & Sons, 1991). Typical amino protecting groups include (C₂H₅O)₂CH—, benzyl represented as Bn, benzyloxycarbonyl represented as Cbz or Z and t-But-O—C(═O)— represented as t-Boc or Boc. In the case of (C₂H₅O)₂CH— or Boc protection, the removal of the amino protecting groups may be carried out under, for example, known acid hydrolysis conditions in a reaction inert solvent, e.g. methanol, ethanol, ethyl acetate, dioxane or mixtures thereof; or without solvent. If desired, the reaction is carried out under acidic conditions, e.g. in the presence of hydrochloric acid or trifluoroacetic acid with a reaction inert scavenger of t-butyl cations, e.g. benzene, thiophenol, anisole, thioanisole, thiocresole, cresole, or dimethyl sulfide. In the case of Bn or Z protection, the removal of the amino protecting groups may be carried out under, for example, known hydrogenolysis conditions in the presence of a metal catalyst, e.g. palladium catalysts such as Pd—C, under hydrogen atmosphere or in the presence of hydrogen sources such as formic acid or ammonium formate in a reaction inert solvent, e.g. methanol, ethanol, ethyl acetate, THF or mixtures thereof. If desired, the reaction is carried out under acidic conditions, e.g. in the presence of hydrochloric acid or acetic acid.

Step 4E

In this Step, a desired compound of formula 4-6 may be prepared from the alcohol compound of formula 4-4, prepared as described in Step 4D in an inert solvent, e.g. aliphatic hydrocarbons, such as hexane, heptane and petroleum ether; aromatic hydrocarbons, such as benzene, toluene o-dichlorobenzene, nitrobenzene, and xylene; and ethers, such as diethyl ether, diisopropyl ether, tetrahydrofuran and dioxane, preferably the ethers.

Step 4F

In this Step, a desired compound of formula 4-7 may be prepared from the compound of formula 4-6, prepared as described in Step 4E in an inert solvent, e.g. aliphatic hydrocarbons, such as hexane, heptane and petroleum ether; aromatic hydrocarbons, such as benzene, toluene o-dichlorobenzene, nitrobenzene, and xylene; and ethers, such as diethyl ether, diisopropyl ether, tetrahydrofuran and dioxane. The reaction may be carried out in the presence of a suitable reducing agent, e.g. tributyltinhydride or triphenyltin hydride. The reaction may be carried out in the presence or absence of a suitable free radical initiator, e.g. 2-2′-azobisisobutylonitrile(AIBN) or (tBuO)2.

Scheme 5:

This illustrates a preparation of an intermediate compound of formula 5-5, which corresponds to the intermediate compound of formula 1-1 wherein the ring A portion contains azole moiety.

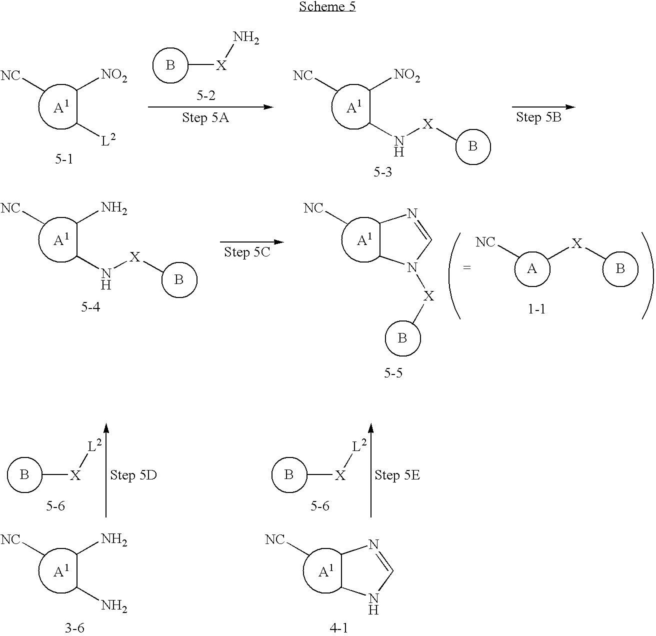

In the above formula, L² represents a halogen atom such as, chlorine, bromine or iodine; and A¹ is defined in Scheme 3.

Step 5A

In this Step, an amino compound of formula 5-3 may be prepared by the amination of a nitro compound of formula 5-1 with the compound of formula 5-2 in an inert solvent. The amination may be carried out in the absence or presence of a base, e.g. in a reaction inert solvent or without solvent. A preferred base is selected from, for example, an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride, or an amine such as triethylamine, tributylamine, diisopropylethylamine, 2,6-lutidine, pyridine or dimethylaminopyridine, in the presence or absence of a reaction inert solvent, e.g. alcohols, such as methanol, ethanol and propanol; benzene, toluene, xylene, o-dichlorobenzene, nitrobenzene, pyridine, dichloromethane, dichloroethane, tetrahydrofuran, dimethylformamide (DMF), dioxane, dimethylsulfoxide (DMSO) or mixtures thereof.

Step 5B

In this Step, a diamine compound of formula 5-4 may be prepared by the reduction of the compound of formula 5-3. This reaction is essentially the same as and may be carried out in the same manner as and using the same reagents and reaction conditions as Step 3B in Scheme 3.

Step 5C

In this Step, the desired imidazole compound of formula 5-5 may be prepared by cyclization of the diamine compound of formula 5-4 with formic acid. This reaction is essentially the same as and may be carried out in the same manner as and using the same reagents and reaction conditions as Step 4A in Scheme 4

Step 5D

In this Step, the compound of formula 5-4 may be prepared from a diamine compound of formula 3-6 with halide agents of formula 5-6 in an inert solvent, e.g. aromatic hydrocarbons, such as benzene, toluene and xylene; halogenated hydrocarbons, such as methylene chloride, chloroform, carbon tetrachloride and dichloroethane; ethers, such as diethyl ether, diisopropyl ether, tetrahydrofuran and dioxane; and pyridine.

Step 5E

In this Step, the desired imidazole compound of formula 5-5 may be prepared by the coupling of a halide compound of formula 5-6 with a N-unsubstituted imidazole compound of formula 4-1 in an inert solvent, e.g. aliphatic hydrocarbons, such as hexane, heptane and petroleum ether; aromatic hydrocarbons, such as benzene, toluene, xylene and nitrobenzene; halogenated hydrocarbons, such as methylene chloride, chloroform, carbon tetrachloride and dichloroethane; ethers, such as diethyl ether, diisopropyl ether, tetrahydrofuran and dioxane; alcohols, such as methanol, ethanol, propanol, isopropanol and butanol; and dimethylformamide (DMF), dimethylsulfoxide (DMSO), 1,3-dimethyl-2-imidazolidinone(DMI) or acetonitrile. This reaction may be carried out in the presence of a base, e.g. an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, potassium carbonate, cesium carbonate, sodium hydride or potassium hydride, or an amine such as triethylamine, tributylamine, diisopropylethylamine, pyridine or dimethylaminopyridine. This reaction may be carried out in the presence of a suitable additive, e.g. tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(I) trifluoromethanesulfonate, copper(II) acetate, copper(II) bromide, copper(II) chloride, copper(II) iodide, copper(II) oxide, 1,10-phenanthroline, dibenzanthracene(DBA) or copper(II) trifluoromethanesulfonate.

Scheme 6:

This illustrates a preparation of an intermediate compound of formula 6-4, which corresponds to the intermediate compound of formula 2-1 wherein the ring A portion contains imidazole moiety.

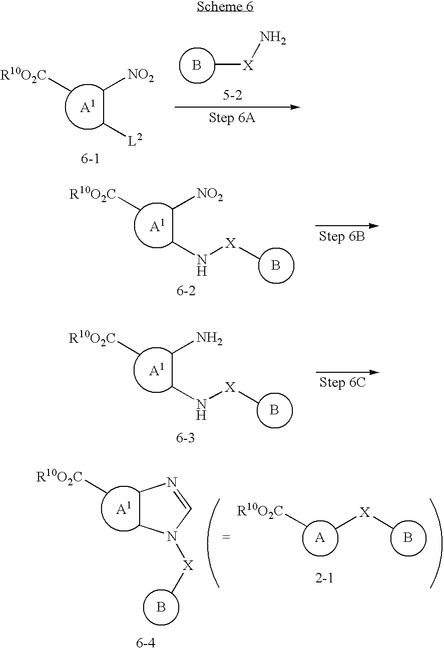

In the above formula, R¹⁰ and A¹ are defined in Scheme 2 and 3 respectively.

Step 6A

In this Step, an amine compound of formula 6-2 may be prepared by the amination of the compound of formula 6-1. This reaction is essentially the same as and may be carried out in the same manner as and using the same reagents and reaction conditions as Step 5A in Scheme 5.

Step 6B

In this Step, a diamine compound of formula 6-3 may be prepared by the reduction of the compound of formula 6-2. This reaction is essentially the same as and may be carried out in the same manner as and using the same reagents and reaction conditions as Step 3B in Scheme 3.

Step 6C

In this Step, the desired imidazole compound of formula 6-4 may be prepared by cyclization of the diamine compound of formula 6-3 with formic acid.

This reaction is essentially the same as and may be carried out in the same manner as and using the same reagents and reaction conditions as Step 4A in Scheme 4.

Scheme 7:

This illustrates the preparation of compounds of formula (Ia) wherein X represents C═O; and formula (Ib) wherein X represents CH—OH.

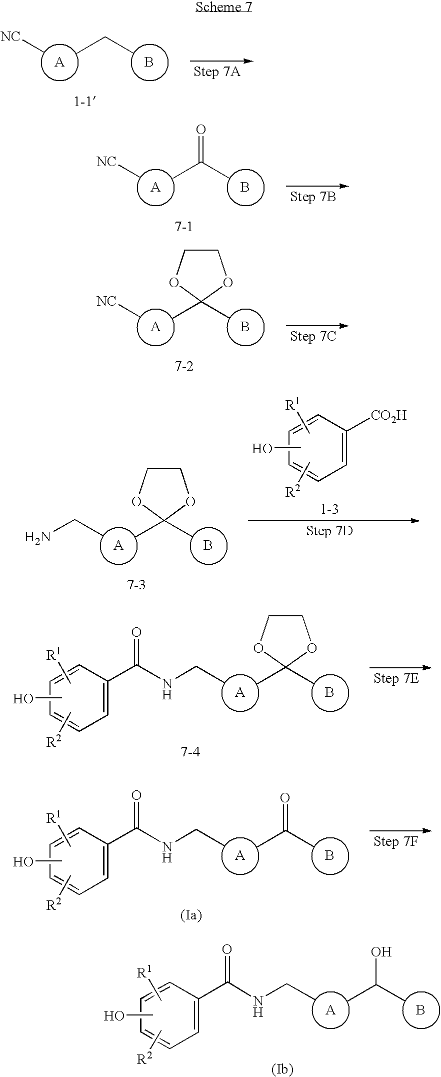

Step 7A

The oxidation can be carried out in the presence of an oxidative agent, e.g. Cr-reagents, such as pyridium chlorochlomate, chromium oxide, pyridium dichlromate; Ru-reagents, such as tetrapropylammonium perruthenate, ruthenium tetraoxide; dimethyl sulfoxide with an activator, such as oxalyl chloride, DCC, sulphortrioxide-pyridine; and dimethyl sulfide with an activator, such as chlorine, N-chlorosuccinimide, in a reaction-inert solvent such as aqueous or non-aqueous organic solvents, e. g. acetic acid, tetrahydrofuran, dioxane, acetone, dimethylformamide, acetonitrile, halogenated hydrocarbons, such as dichloromethane, dichloroethane, chloroform.

Step 7B

In this Step, an acetal compound of formula 7-2 can be prepared by the protection of a ketone compound of formula 7-1 in the presence or the absence of a catalyst, e.g. sulfonic acids, such as p-toluenesulfonic acid and benzenesulfonic acid, in a reaction-inert solvent, e.g. aromatic hydrocarbons, such as benzene, toluene and xylene; ethers, such as tetrahydrofuran or dioxane; acetone; dimethylformamide; halogenated hydrocarbons, such as dichloromethane, dichloroethane or chloroform. The steps may be carried out by using, for example, the compound of formula 7-1, appropriate ethylene glycol or propylene glycol, using the methods described in Protective Groups in Organic Synthesis edited by T. W. Greene et al. (John Wiley & Sons, 1991).

Step 7C

In this Step, an amine compound of formula 7-3 may be prepared by the reduction of the compound of formula 7-2. This reaction is essentially the same as and may be carried out in the same manner as and using the same reagents and reaction conditions as Step 1A in Scheme 1.

Step 7D

In this Step, an amide compound of formula 7-4 can be prepared by the coupling reaction of an amine compound of formula 7-3 with an acid compound of formula 1-3 in the presence or absence of a coupling reagent in an inert solvent. This reaction is essentially the same as and may be carried out in the same manner as and using the same reagents and reaction conditions as Step 1B in Scheme 1.

Step 7E

In this Step, a ketone compound of formula (Ia) can be prepared by the hydrolysis reaction of a ketal compound of formula 7-4 in the presence or the absence of a catalyst, e.g. hydrogen halides, such as hydrogen chloride and hydrogen bromide; sulfonic acids, such as p-toluenesulfonic acid and benzenesulfonic acid; ammonium salts, such as pyridium p-toluenesulfonate and ammonium chloride; and carboxylic acid, such as acetic acid and trifluoroacetic acid in a reaction-inert solvent, e.g. alcohols, such as methanol or ethanol; ethers, such as tetrahydrofuran or dioxane; acetone; dimethylformamide; halogenated hydrocarbons, such as dichloromethane, dichloroethane or chloroform; acids, such as acetic acid, hydrogen chloride, hydrogen bromide and sulfuric acid.

Step 7F

In this Step, an alcohol compound of formula (Ib) can be prepared by the reduction of a ketone compound of formula (Ia) with a reducing agent, e.g. NaBH₄, LiAlH₄, LiBH₄, or ZnBH₄ in an inert solvent, e.g. methanol, ethanol, diglyme, or mixtures thereof.

Scheme 8:

This illustrates a preparation of intermediate compound of formula 8-3, which corresponds to the intermediate compound of formula 2-1 wherein the ring A portion contains oxazole moiety.

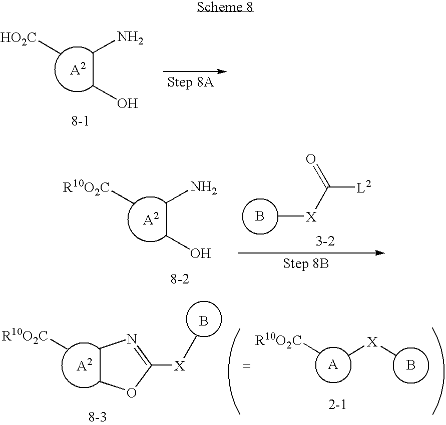

In the above formula, A² represents a monocyclic, aromatic, saturated or partially unsaturated heterocyclic or carbocyclic group having from 5 to 9 ring atoms; said heterocyclic group contains either from 1 to 3 nitrogen atoms, or 1 nitrogen atoms and/or 1 or 2 oxgen or sulfur atoms; said heterocyclic or carbocyclic group are unsubstituted or are substituted by at least one substituent selected from the group consisting of substituents α; said substituents α are selected from the group consisting of halogen atoms, alkyl groups having from 1 to 6 carbon atoms, alkoxy groups having from 1 to 6 carbon atoms, cyano groups, alkanoyl groups having from 1 to 6 carbon atoms, haloalkyl groups having from 1 to 6 carbon atoms, oxo groups or haloalkoxy groups having from 1 to 6 carbon atoms;

Examples of said heterocyclic or carbocyclic group include, but are not limited to, cyclopentane, cyclopentene, cyclohexane, cyclohexene, phenyl, cycloheptane, cycloheptene, pyrrole, thiophene, furan, imidazole, pyrazole, thiazole, oxazole, pyridine, pyrazine, pyrimidine, pyridazine, piperidine, piperazine or morpholine. R¹⁰ is defined in Scheme 2. L² is defined in Scheme 3.

Step 8A

In this Step, an ester compound of formula 8-2 can be prepared by the esterification of an acid compound of formula 8-1.

The esterification may be carried out by a number of standard procedures known to those skilled in the art (e.g., Protective Groups in Organic Synthesis, Third edition. ed. T. W. Green and P. G. M. Wuts, Wiley-Interscience., pp 373-377.). Typical esterification can be carried out in the presence of an acid catalyst, e.g. sulfuric acid, p-toluenesulfonic acid, camphorsulfonic acid and benzenesulfonic acid, in a suitable reaction-inert solvent, e.g. methanol or ethanol. Typical esterification can also be carried out with a suitable C₁₋₆ alkylhalide or benzylhalide in the presence of a base, K₂CO₃, Cs₂CO3, NaHCO₃ and DBU, in a suitable reaction-inert solvent, e.g. ethers such as tetrahydrofuran, 1,2-dimethoxyethane, diethyl ether, diisopropyl ether, diphenyl ether, DMF, DMSO, R′OH and 1,4-dioxane. The esterification also carried out with trimethylsilyldiazomethane in a suitable reaction-inert solvent, e.g. methanol, benzene and toluene. The esterification also carried out with diazomethane in a suitable reaction-inert solvent, e.g. diethyl ether. Alternatively, the esterification may be carried out with R′OH, in the presence of a coupling agent, e.g. DCC, WSC, diisoproopylcyanophosphonate (DIPC), BOPCl and 2,4,6-trichlorobenzoic acid chloride, and a tertiaryamine, e.g. i-Pr₂Net or Et₃N, in a suitable solvent, e.g. DMF, THF, diethyl ether, DME, dichloromethane and DCE.

Step 8B

In this Step, an oxazole compound of formula 8-2 may be prepared by the cyclization of the amino compound of formula 8-2 under conditions known to those skilled in the art. This reaction is essentially the same as and may be carried out in the same manner as and using the same reagents and reaction conditions as Step 3A in Scheme 3.

Scheme 9:

This illustrates a preparation of intermediate compound of formula 9-3, which corresponds to the intermediate compound of formula 1-1 wherein the ring A portion contains indazole moiety.

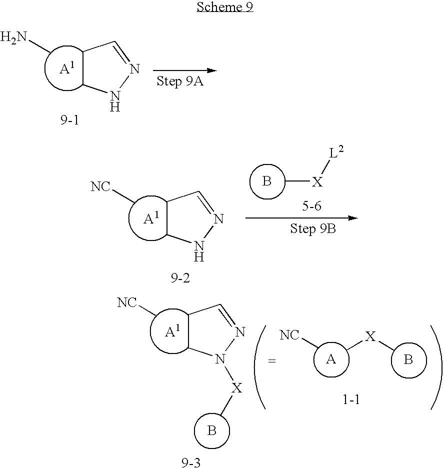

In the above formula, A¹ and L² are defined in Scheme 3.

Step 9A

In this Step, a cyano compound of formula 9-2 can be prepared from an amino compound of formula 9-1 through Sandmeyer's reaction under conditions known to those skilled in the art. The amino compound of formula 9-1 may be first subjected to diazotization of the amine portion, followed by cyanidation to afford a corresponding the cyano compound of formula 9-2. This diazotization may be carried out in the presence sodium nitrite and in the presence of a solvent, e.g. H₂O, aqueous HCl, or aqueous H₂SO₄. This diazotization may be carried out in the presence of an acid, e.g. hydrochloric acid or acetic acid. This cyanidation may be carried out in the presence cyanide, e.g. copper(I) cyanide or sodium cyanide. The cyanidation may be normally and preferably effected in the presence of a solvent, e.g. H₂O, aqueous HCl, aqueous H₂SO₄.

Step 9B

In this Step, the desired indazole compound of formula 9-3 may be prepared by the coupling of a halide compound of formula 5-6 with a N-unsubstituted indazole compound of formula 9-2 in an inert solvent. This reaction is essentially the same as and may be carried out in the same manner as and using the same reagents and reaction conditions as Step 5E in Scheme 5.

Scheme 10:

This illustrates a preparation of intermediate compounds of formula 10-3 and 10-4 which correspond to the intermediate compound of formula 2-1 wherein the ring A portion contains indazole moiety.

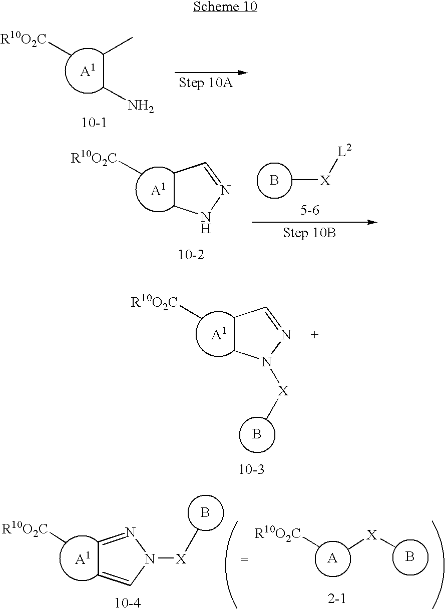

In the above formula, R¹⁰ is defined in Scheme 2; and A¹ and L² are defined in Scheme 3.

Step 10A

In this Step, an indazole compound of formula 10-2 can be prepared from an amino compound of formula 10-1 through reaction under conditions known to those skilled in the art. (D. B. Batt, et al., J. Med. Chem. 2000, 46, 41-58). The amino compound of formula 9-1 may be first subjected to diazotization of the amine portion, followed by cyclization to afford a corresponding the indazole compound of formula 10-2. The diazotization is essentially the same as and may be carried out in the same manner as and using the same reagents and reaction conditions as Step 9A in Scheme 9. In this step, this reaction can be carried out in the presence of ammonium tetrafluoroborate. The cyclization may be carried out in the presence of a base, e.g. potassium acetate. This cyclization may be carried out in the presence catalyst, e.g. 18-Crown-6 or 15-Crown-5. The cyclization may be normally and preferably effected in the presence of a solvent, e.g. halogenated hydrocarbons, such as dichloromethane, dichloroethane or chloroform, acids, such as acetic acid, aqueous H₂SO₄, aqueous HCl, alcohols, such as methanol or ethanol.

Step 10B

In this Step, the desired indazole compound of formula 10-3 and 10-4 may be prepared by the coupling of a halide compound of formula 5-6 with a N-unsubstituted indazole compound of formula 10-2 in an inert solvent. This reaction is essentially the same as and may be carried out in the same manner as and using the same reagents and reaction conditions as Step SE in Scheme 5.

Scheme 11:

This illustrates a preparation of compound of formula (Ic), which corresponds to the intermediate compound of formula 2-1 wherein the ring A portion contains oxazole moiety.

This illustrates the preparation of compounds of formula (Ic) wherein X represents CH₂ and the ring A portion contains pyridine moiety; and formula (Ib) wherein X represents (CH₂)₂ and the ring A portion contains pyridine moiety.

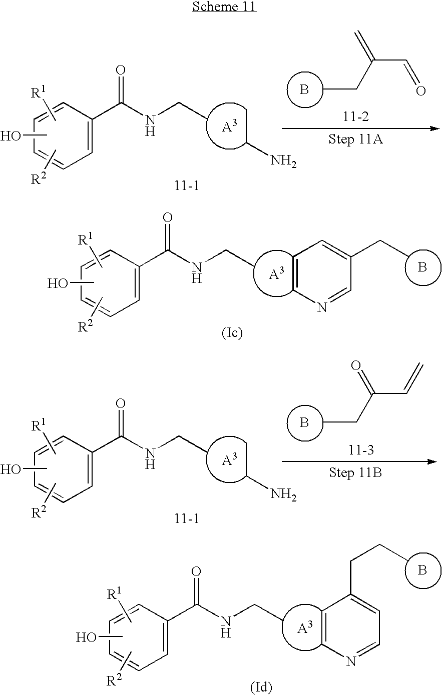

In the above formula, A³ represents a monocyclic, aromatic, saturated or partially unsaturated heterocyclic or carbocyclic group having from 5 to 9 ring atoms; said heterocyclic group contains either from 1 to 3 nitrogen atoms, or 1 nitrogen atoms and/or 1 or 2 oxgen or sulfur atoms; said heterocyclic or carbocyclic group are unsubstituted or are substituted by at least one substituent selected from the group consisting of substituents α; said substituents α are selected from the group consisting of halogen atoms, alkyl groups having from 1 to 6 carbon atoms, alkoxy groups having from 1 to 6 carbon atoms, cyano groups, alkanoyl groups having from 1 to 6 carbon atoms, haloalkyl groups having from 1 to 6 carbon atoms, oxo groups or haloalkoxy groups having from 1 to 6 carbon atoms;

Examples of said heterocyclic or carbocyclic group include, but are not limited to, cyclopentane, cyclopentene, cyclohexane, cyclohexene, phenyl, cycloheptane, cycloheptene, pyrrole, thiophene, furan, imidazole, pyrazole, thiazole, oxazole, pyridine, pyrazine, pyrimidine, pyridazine, piperidine, piperazine or morpholine.

Step 11A

In this Step, fused-pyridine compound of formula (Ic) may be prepared by the cyclization of the amino compound of formula 11-1 with an enone compound of formula 11-2. The reaction may be carried out in the presence or absence of a solvent, e.g. alcohols, such as methanol, ethanol and propanol, dimethylformamide, halogenated hydrocarbons, such as dichloromethane, dichloroethane or chloroform. This reaction may be carried out in the presence or absence of an acid, e.g. nitrobenzenesulfonic acid, hydrochloric acid and acetic acid or sulfuric acid. This reaction may be carried out in the presence or absence of a catalyst, e.g. zinc chloride or aluminum oxide.

Step 11B

In this Step, a fused-pyridine compound of formula (Id) may be prepared by the cyclization of the amino compound of formula 11-2 with an enone compound of formula 11-3. This reaction is essentially the same as and may be carried out in the same manner as and using the same reagents and reaction conditions as Step 11A in Scheme 11.

Scheme 12:

This illustrates a preparation of an intermediate compound of formula 11-1.

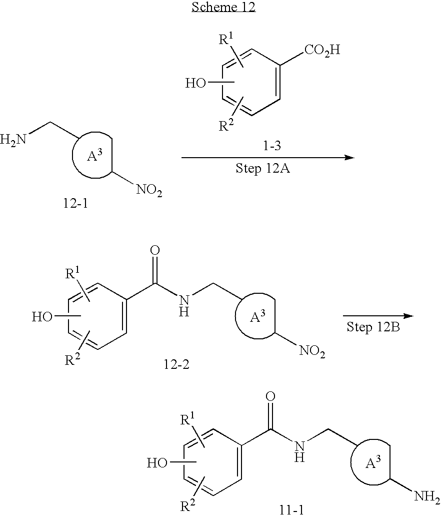

In the above formula, A³ is defined in Scheme 11.

Step 12A

In this Step, an amide compound of formula 12-2 may be prepared by the coupling of the amino compound of formula 12-1 with an acid compound of formula 1-3. This reaction is essentially the same as and may be carried out in the same manner as and using the same reagents and reaction conditions as Step 1B in Scheme 1.

Step 12B

In this Step, an amine compound of formula 11-1 may be prepared by the reduction of the nitro compound of formula 12-2. This reaction is essentially the same as and may be carried out in the same manner as and using the same reagents and reaction conditions as Step 3B in Scheme 3.

The starting materials in the aforementioned general syntheses may be commercially available or obtained by conventional methods known to those skilled in the art.

In the above Schemes from 1 to 12, examples of suitable solvents include a mixture of any two or more of those solvents described in each Step.

The compounds of formula (I), and the intermediates above-mentioned preparation methods can be isolated and purified by conventional procedures, such as recrystallization or chromatographic purification.

The optically active compounds of this invention can be prepared by several methods. For example, the optically active compounds of this invention may be obtained by chromatographic separation, enzymatic resolution or fractional crystallization from the final compounds.

### Method for Assessing Biological Activities

NR2B Binding Assay

The activity of the bicyclic amide compounds of the present invention, as NR2B antagonists, is determined by their ability to inhibit the binding of NR2B subunit at its receptor sites employing radioactive ligands.

The NR2B antagonist activity of the bicyclic amide compounds is evaluated by using the standard assay procedure described in, for example, J. Pharmacol., 331, pp117-126, 1997. This method essentially involves determining the concentration of the individual compound required to reduce the amount of radiolabelled NR2B ligands by 50% at their receptor sites, thereby affording characteristic IC₅₀ values for each compound tested. More specifically, the assay is carried out as follows.

Membranes were prepared by homogenization of forebrain of male CD rats weighing between 170˜190 g by using glass-Teflon homogenizer in 0.32 M sucrose at 4° C. The crude nuclear pellet was removed by centrifugation at 1000×g for 10 min, and the supernatant centrifuged at 17000×g for 25 min. The resulting pellet was resuspended in 5 mM Tris acetate pH 7.4 at 4° C. for 10 min to lyse cellular particles and again centrifuged at 17000×g. The resulting pellet (P2 membrane) was washed twice in Tris acetate, resuspended at 5.5 mg protein/ml and stored at −20° C. until use. All the manipulation was done on ice, and stock solution and equipment were kept on ice at all time.

For the saturation assay, receptor saturation was determined by incubating [³H]-1-[(1S*,2S*)-2-hydroxy-2-(4-hydroxyphenyl)-1-methylethyl]-4-phenylpiperidin-4-ol and 50 μg protein of P2 membrane for 60 minutes at room temperature in a final 100 μl of incubation buffer (50 mM Tris HCl, pH7.4). Total and non-specific bindings (in the presence of 10 μM of unlabeled 1-[(1S*,2S*)-2-hydroxy-2-(4-hydroxyphenyl)-1-methylethyl]-4-phenylpiperidin-4-ol) were determined in a range of [3H]-1-[(1S*,2S*)-2-hydroxy-2-(4-hydroxyphenyl)-1-methylethyl]-4-phenylpiperidin-4-ol concentrations (0.625 nM to 60 nM). [³H]-1-[(1S*,2S*)-2-hydroxy-2-(4-hydroxyphenyl)-1-methylethyl]-4-phenylpiperidin-4-ol is as follows:

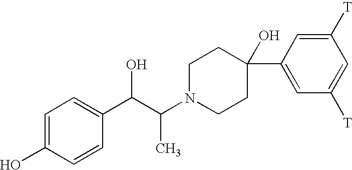

(wherein T is tritio (³H)).

For the competition assay, test compounds were incubated in duplicate with 5 nM [³H]-1-[(1S*,2S*)-2-hydroxy-2-(4-hydroxyphenyl)-1-methylethyl]-4-phenylpiperidin-4-ol and 50 μg protein of P2 membrane for 60 minutes at room temperature in a final 100 μl of 50 mM Tris HCl buffer (pH7.4). Nonspecific binding was determined by 10 μM of unlabeled 1-[(1S*,2S*)-2-hydroxy-2-(4-hydroxyphenyl)-1-methylethyl]-4-phenylpiperidin-4-ol (25 μl). The saturation derived K_{D} gained in saturation assay was used for all Ki calculations.

All incubations were terminated by rapid vacuum filtration over 0.2% polyethyleneimine soaked Whatman GF/B glass fibre filter paper using a SKATRON cell harvester followed by three washes with ice-cold filtration buffer (5 mM Tris HCl, pH 7.4.). Receptor-bound radioactivity was quantified by liquid scintillation counting using Packard LS counter. Competition assays were performed by counting Wallac GF/B filters on Betaplate scintillation counter (Wallac).

The compound prepared in the working example 8 as described below was tested by this method, and showed a Ki value of 2 nM with respect to binding affinity for the NR2B receptor. In this test, the compounds of the present invention exhibited excellent binding activity for the NR2B receptor.

Human NR2B Cell Functional Assay

HEK293 cells stably expressing human NR1b/2B receptor were used for cell functional assay. Cells were grown in 75-cm² culture flasks, using Dulbecco's modified Eagle's medium (DMEM, high glucose) supplemented with 10% fetal bovine, 52 μg/ml Zeocin, 530 μg/ml Geneticin, 100 units/ml penicillin and 100 μg/ml streptomycin. Cells were maintained in a humidified atmosphere in 5% CO₂ at 37° C., and 50-60% confluent cells were harvested by 0.05% trypsin containing 0.53 mM EDTA. The day before the experiment, expression of NR1b/2B receptor was induced by 5 μM ponasteron A in DMEM (40 ml) in the presence of 400 μM ketamine to prevent excitotoxicity. The induction was performed for 19-24 hours, using 50-60% confluent cells.

Cells were washed with 10 ml of Ca²⁺-free Krebs-Ringer Hepes buffer (KRH) containing 400 μM ketamine, and the loading of 5 μM fura-2 acetoxymethyl ester was made for 2 hrs at room temperature in the presence of 400 μM ketamine in Ca²⁺-free KRH (10 ml). Subsequently, cells were collected in 50 ml tube by pipetting manipulation and centrifuged at 850 rpm for 2 min. Supernatant was removed, and cells were washed with 10 ml of Ca²⁺-free KRH buffer, followed by centrifugation again. This manipulation was repeated 4 times to remove ketamine, glutamate and glycine. Cells were re-suspended in Ca²⁺-free KRH buffer, and 50 μl of cell suspension was added to each well of 96-well plates at a density of 100,000 cells/well, followed by adding test compounds dissolved in 50 μl of Ca²⁺-free KRH. After pre-incubation for 30 min, agonists (final 100 μM glutamic acid and 10 μM glycine) dissolved in 25 μl of KRH containing 9 mM Ca²⁺ (final 1.8 mM) were added. Fura-2 fluorescence (excitation wavelengths: 340 nm and 380 nm; emission wavelengths 510-520 nm) was monitored with a fluorescence imaging system, FDSS6000. The Δ fluorescence ratio F340/F380 (i.e., the fluorescence ratio immediately post-agonist—the basal fluorescence ratio; calculated as AUC) was used for evaluation of drug effects on agonists-induced changes in intracellular Ca²⁺. The basal fluorescence ratio was determined in the presence of 10 μM MK-801.

Rat Haloperidol-Induced Catalepsy Assay:

Fasted male CD rats were used (7-8 weeks old). Test compound or vehicle was given subcutaneously then haloperidol 0.5 mg/kg s.c. Sixty minutes after haloperidol-injection, the duration of catalepsy was quantified by placing the animals forepaws on an elevated bar and determining the latency to remove both forepaws from the bar. The cutoff latency was 60 seconds. Experimenter was blind to treatments during testing.

Human Dofetilide Binding

Human HERG transfected HEK293S cells were prepared and grown in-house. The collected cells were suspended in 50 mM Tris-HCl (pH 7.4 at 4° C.) and homogenized using a hand held Polytron PT 1200 disruptor set at full power for 20 sec on ice. The homogenates were centrifuged at 48,000×g at 4° C. for 20 min. The pellets were then resuspended, homogenized, and centrifuged once more in the same manner. The final pellets were resuspended in an appropriate volume of 50 mM Tris-HCl, 10 mM KCl, 1 mM MgCl₂ (pH 7.4 at 4° C.), homogenized, aliquoted and stored at −80° C. until use. An aliquot of membrane fractions was used for protein concentration determination using BCA protein assay kit (PIERCE) and ARVOsx plate reader (Wallac). Binding assays were conducted in a total volume of 200 μl in 96-well plates. Twenty μl of test compounds were incubated with 20 μl of [³H]-dofetilide (Amersham, final 5 nM) and 160 μl of membrane homogenate (25 μg protein) for 60 minutes at room temperature. Nonspecific binding was determined by 10 μM dofetilide at the final concentration. Incubation was terminated by rapid vacuum filtration over 0.5% presoaked GF/B Betaplate filter using Skatron cell harvester with 50 mM Tris-HCl, 10 mM KCl, 1 mM MgCl₂, pH 7.4 at 4° C. The filters were dried, put into sample bags and filled with Betaplate Scint. Radioactivity bound to filter was counted with Wallac Betaplate counter.

I_{HERG} Assay

HEK 293 cells which stably express the HERG potassium channel were used for electrophysiological study. The methodology for stable transfection of this channel in HEK cells can be found elsewhere (Z. Zhou et al., 1998, Biophysical journal, 74, pp230-241). Before the day of experimentation, the cells were harvested from culture flasks and plated onto glass coverslips in a standard MEM medium with 10% FCS. The plated cells were stored in an incubator at 37° C. maintained in an atmosphere of 95%O₂/5%CO₂. Cells were studied between 15-28 hrs after harvest.

HERG currents were studied using standard patch clamp techniques in the whole-cell mode. During the experiment the cells were superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings was made using a patch clamp amplifier and patch pipettes which have a resistance of 1-3MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15 MΩ and seal resistances >1 GΩ was accepted for further experimentation. Series resistance compensation was applied up to a maximum of 80%. No leak subtraction was done. However, acceptable access resistance depended on the size of the recorded currents and the level of series resistance compensation that can safely be used. Following the achievement of whole cell configuration and sufficient for cell dialysis with pipette solution (>5min), a standard voltage protocol was applied to the cell to evoke membrane currents. The voltage protocol is as follows. The membrane was depolarized from a holding potential of −80 mV to +20 mV for 1000 ms. This was followed by a descending voltage ramp (rate 0.5 mV msec⁻¹) back to the holding potential. The voltage protocol was applied to a cell continuously throughout the experiment every 4 seconds (0.25 Hz). The amplitude of the peak current elicited around −40 mV during the ramp was measured. Once stable evoked current responses were obtained in the external solution, vehicle (0.5% DMSO in the standard external solution) was applied for 10-20 min by a peristalic pump. Provided there were minimal changes in the amplitude of the evoked current response in the vehicle control condition, the test compound of either 0.3, 1, 3, 10 μM was applied for a 10 min period. The 10 min period included the time which supplying solution was passing through the tube from solution reservoir to the recording chamber via the pump. Exposing time of cells to the compound solution was more than 5 min after the drug concentration in the chamber well reached the attempting concentration. There reversibility. Finally, the cells was exposed to high dose of dofetilide (5 μM), a specific IKr blocker, to evaluate the insensitive endogenous current.

All experiments were performed at room temperature (23±1° C.). Evoked membrane currents were recorded on-line on a computer, filtered at 500-1 KHz (Bessel −3 dB) and sampled at 1-2 KHz using the patch clamp amplifier and a specific data analyzing software. Peak current amplitude, which occurred at around −40 mV, was measured off line on the computer.

The arithmetic mean of the ten values of amplitude was calculated under control conditions and in the presence of drug. Percent decrease of I_{N} in each experiment was obtained by the normalized current value using the following formula: I_{N}=(1−I_{D}/I_{C})×100, where I_{D} is the mean current value in the presence of drug and I_{C} is the mean current value under control conditions. Separate experiments were performed for each drug concentration or time-matched control, and arithmetic mean in each experiment is defined as the result of the study.

Mice PSL Method

Surgery of partial sciatic nerve ligation (PSL) was made according to Seltzer et al. (Pain 43, 1990, 205-218). Von Fray hair test was applied slowly to the plantar surface of the hind operated paw until the hairs bent. Each hair was tested 10 times in ascending order of force to different loci of the paw with one to two second intervals between each application. Once a withdrawal response was established, the paw was re-tested with the same hair. The lowest amount of force required to elicit a response was recorded as the paw-withdrawal threshold, measured in grams.

Serum protein binding

Serum protein binding of NR2B topic compounds (1 uM) in humans and ddY mice were measured in method of equilibrium dialysis using 96-well plate type equipment. Spectra-Por® regenerated cellulose membranes (molecular weight cut-off 12,000-14,000, 12 mm×120 mm) was soaked for over night in distilled water, then for 20 minutes in 30% ethanol, and finally for 15 minutes in dialysis buffer (0.10 M PBS: phosphate buffered saline, pH 7.4). Fresh humans and ddY mice serum (20 ml each) was prepared. The dialysis was assembled with being careful not to puncture or tear the membranes and added 150 ul of serum to one side of each well and 150 ul of dialysis buffer to the other side of each well. After 4 hours incubation at 37° C. for 60 r.p.m, remove the serum and buffer samples and an aliquot of collected serum and buffer samples were mixed for buffer and serum at following rates:

1) 40 ul serum samples were mixed with 120 ul buffer

2) 120 ul buffer samples were mixed with 40 ul serum

Then, mixed samples were extracted with 600 μl acetonitrile containing (2R,3R)-2-(diphenylmethyl)-N-(2-methoxybenzyl)quinuclidin-3-amine at 25 ng/ml (as HPLC-MS-MS internal standard) and measured in LC/MS/MS analysis.

Calculations

The fraction of substrate unbound, fᵤ=1−{([plasma]_{eq}−[buffer])/([plasma]_{eq})}

where [plasma]_{eq} and [buffer]_{eq} are the concentrations of substrate in plasma and buffer, respectively.

Aqueous Solubility

Aqueous solubility in the mediums (a)-(c) was determined by method (1) or (2). (1) Vials containing approx. 1 mg of compound and 1 mL of each medium were agitated for 24 hours at room temperature. Insoluble materials were removed by centrifugation at 10,000 rpm for 10 minutes twice. The supernatants were assayed by HPLC. (2) Whatman Mini-UniPrep chambers (Clifton, N.J., USA) containing more than 0.5 mg of compound and 0.5 mL of each medium were shaken overnight (over 8 hours) at room temperature. All samples were filtered through a 0.45 μm PVDF membrane into a Whatman Mini-UniPrep plunger before analysis. The filtrates were assayed by HPLC.

<Mediums>

(a) Simulated gastric fluid with no enzyme (SGN) at pH 1.2: Dissolve 2.0 g of NaCl in 7.0 mL of 10N HCl and sufficient water to make 1000 mL.

(b) Phosphate buffered saline (PBS) at pH 6.5: Dissolve 6.35 g of KH₂PO₄, 2.84 g of Na₂HPO₄ and 5.50 g of NaCl in sufficient water to make 1000 mL, adjusting the pH of this solution to 6.5.

(c) Water for injection (WFI).

Pharmaceutically acceptable salts of the compounds of formula (1) include the acid addition and base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection, and Use, Wiley-VCH, Weinheim, Germany (2002). A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D₂O, d₆-acetone, d₆-DMSO.

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds of formula (I) include references to salts thereof and to solvates and clathrates of compounds of formula (I) and salts thereof. The invention includes all polymorphs of the compounds of formula (I) as hereinbefore defined.

Also within the scope of the invention are so-called “prodrugs” of the compounds of formula (I). Thus certain derivatives of compounds of formula (I) which have little or no pharmacological activity themselves can, when metabolised upon administration into or onto the body, give rise to compounds of formula (I) having the desired activity. Such derivatives are referred to as “prodrugs”.

Prodrugs in accordance with the invention can, for example, be produced by replacing appropriate functionalities present in the compounds of formula (I) with certain moieties known to those skilled in the art as “pro-moieties” as described, for example, in “Design of Prodrugs” by H Bundgaard (Elsevier, 1985).

Finally, certain compounds of formula (I) may themselves act as prodrugs of other compounds of formula (I).

Compounds of formula (I) containing one or more asymmetric carbon atoms can exist as two or more optical isomers. Where a compound of formula (I) contains an alkenyl or alkenylene group, geometric cis/trans (or Z/E) isomers are possible, and where the compound contains, for example, a keto or oxime group, tautomeric isomerism (‘tautomerism’) may occur. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof.

Cis/trans isomers may be separated by conventional techniques well known to those skilled in the art, for example, fractional crystallization and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystallization of diastereoisomeric salts formed by reaction of the racemate with a suitable optically active acid or base, for example, tartaric acid.

The present invention also includes all pharmaceutically acceptable isotopic variations of a compound of formula (I). An isotopic variation is defined as one in which at least one atom is replaced by an atom having the same atomic number, but an atomic mass different from the atomic mass usually found in nature.

Examples of isotopes suitable for inclusion in the compounds of the invention include isotopes of hydrogen, such as 2H and 3H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the compounds of the invention with isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the compounds of formula (I) can generally be prepared by conventional techniques known to those skilled in the art or by processes analogous to those described in the accompanying Examples and Preparations using appropriate isotopic variations of suitable reagents.

The compounds of formula (I) may be freeze-dried, spray-dried, or evaporatively dried to provide a solid plug, powder, or film of crystalline or amorphous material. Microwave or radio frequency drying may be used for this purpose.

The compounds of the invention may be administered alone or in combination with other drugs and will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds of the invention may be administered in combination, separately, simultaneously or sequentially, with one or more other pharmacologically active agents. Suitable agents, particularly for the treatment of pain, include:

(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;

(ii) nonsteroidal antiinflammatory drugs (NSAIDs), e.g. aspirin, diclofenac, diflusinal, etodolac, fenbufen, fenoprofen, flufenisal, flurbiprofen,ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;

(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;

(iv) benzodiazepines having a sedative action, e.g. chlordiazepoxide, clorazepate, diazepam, flurazepam, lorazepam, oxazepam, temazepam, triazolam and their pharmaceutically acceptable salts,

(v) H₁ antagonists having a sedative action, e.g. diphenhydramine, pyrilamine, promethazine, chlorpheniramine, chlorcyclizine and their pharmaceutically acceptable salts;

(vi) miscellaneous sedatives such as glutethimide, meprobamate, methaqualone, dichloralphenazone and their pharmaceutically acceptable salts;

(vii) skeletal muscle relaxants, e.g. baclofen, carisoprodol, chlorzoxazone, cyclobenzaprine, methocarbamol, orphrenadine and their pharmaceutically acceptable salts,

(viii) alpha-2-delta ligands, e.g. gabapentin and pregabalin;

(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl) quinazoline;

(x) tricyclic antidepressants, e.g. desipramine, imipramine, amytriptiline and nortriptiline;

(xi) anticonvulsants, e.g. carbamazepine and valproate;

(xii) serotonin reuptake inhibitors, e.g. fluoxetine, paroxetine, citalopram and sertraline;

(xiii) mixed serotonin-noradrenaline reuptake inhibitors, e.g. milnacipran, venlafaxine and duloxetine;

(xiv) noradrenaline reuptake inhibitors, e.g. reboxetine;

(xv) Tachykinin (NK) antagonists, particularly Nk-3, NK-2 and NK-1 antagonists, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)

(xvi) Muscarinic antagonists, e.g oxybutin, tolterodine, propiverine, tropsium chloride and darifenacin;

(xvii) COX-2 inhibitors, e.g. celecoxib, rofecoxib and valdecoxib;

(xviii) Non-selective COX inhibitors (preferably with GI protection), e.g. nitroflurbiprofen (HCT-1026);

(xix) coal-tar analgesics, in particular, paracetamol;

(xx) neuroleptics, such as droperidol;

(xxi) Vanilloid receptor agonists, e.g. resinferatoxin;

(xxii) Beta-adrenergic compounds such as propranolol;

(xxiii) Local anaesthetics, such as mexiletine;

(xxiv) Corticosteriods, such as dexamethasone

(xxv) serotonin receptor agonists and antagonists;

(xxvi) cholinergic (nicotinic) analgesics; and

(xxvii) miscellaneous analgesic agents, such as Tramadol®.

Thus, the invention further provides a combination comprising a compound of the invention or a pharmaceutically acceptable salt, solvate or pro-drug thereof, and a compound or class of compounds selected from the group (i)-(xxvii), above. There is also provided a pharmaceutical composition composition comprising such a combination, together with a pharmaceutically acceptable excipient, diluent or carrier, particularly for the treatment of a disease for which an alpha-2-delta ligand is implicated.

Combinations of the compounds of the present invention and other therapeutic agents may be administered separately, sequentially or simultaneously. Thus, the present invention extends to a kit comprising a compound of the invention, one or more other therapeutic agents, such as those listed above, and a suitable container.

The compounds of the present invention may be formulated by any convenient means using well-known carriers and excipients. Thus, the present invention also provides a pharmaceutical composition comprising a compound of the invention or a pharmaceutically acceptable ester or a pharmaceutically acceptable salt thereof with one or more pharmaceutically acceptable carriers.

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Compound of formula (I) | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

* Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

Topical Administration

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff”.

Formulations for inhaled/intranasal administration may be formulated to be immediate and/or modified release. Modified release formulations include delayed-, sustained-, pulsed-, controlled dual-, targeted and programmed release.

Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

Ocular/Andial Administration

The compounds of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The compounds of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

The compounds of the invention can be administered via either the oral, parenteral or topical routes to mammals. In general, these compounds are most desirably administered to humans in doses ranging from 0.1 mg to 3000 mg, preferably from 1 mg to 500 mg, which may be administered in a single dose or in divided doses throughout the day, although variations will necessarily occur depending upon the weight and condition of the subject being treated, the disease state being treated and the particular route of administration chosen.

These dosages are based on an average human subject having a weight of about 65 to 70 kg. The physician will readily be able to determine doses for subjects whose weight falls outside this range, such as infants and the elderly.

For example, a dosage level that is in the range of from 0.01 mg to 10 mg per kg of body weight per day is most desirably employed for treatment of pain associated with inflammation.

### EXAMPLES

The invention is illustrated in the following non-limiting examples in which, unless stated otherwise: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (tlc) and reaction times are given for illustration only; melting points (m.p.) given are uncorrected (polymorphism may result in different melting points); the structure and purity of all isolated compounds were assured by at least one of the following techniques: tlc (Merck silica gel 60 F₂₅₄ precoated TLC plates or Merck NH₂ F₂₅₄ₛ precoated HPTLC plates), mass spectrometry, nuclear magnetic resonance (NMR), infrared red absorption spectra (IR) or microanalysis. Yields are given for illustrative purposes only. Flash column chromatography was carried out using Merck silica gel 60 (230-400 mesh ASTM) or Fuji Silysia Chromatorex® DU3050 (Amino Type, 30˜50 μm). Low-resolution mass spectral data (EI) were obtained on a Automass 120 (JEOL) mass spectrometer. Low-resolution mass spectral data (ESI) were obtained on a Quattro II (Micromass) mass spectrometer. NMR data were determined at 270 MHz (JEOL JNM-LA 270 spectrometer) or 300 MHz (JEOL JNM-LA300) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br.=broad, etc. IR spectra were measured by a Shimazu infrared spectrometer (IR-470). Optical rotations were measured using a JASCO DIP-370 Digital Polarimeter (Japan Spectroscopic CO, Ltd.).

Chemical symbols have their usual meanings; b.p. (boiling point), m.p. (melting point), l (liter(s)), ml (milliliter(s)), g (gram(s)), mg(milligram(s)), mol (moles), mmol (millimoles), eq. (equivalent(s)).

### Example 1

### N-[(2-benzyl-1H-benzimidazol-5-yl)methyl]-4-hydroxybenzamide

### A. N-(4-cyano-2-nitrophenyl)-2-phenylacetamide

A mixture of 4-amino-3-nitrobenzonitrile (2 g, 12.2 mmol) and phenylacetyl chloride (1.6 ml, 12.2 mmol) in toluene (130 ml) was refluxed overnight. To the mixture was added 2 N aqueous NaOH (100 ml) and the whole was extracted with ethyl acetate (200 ml×2). The combined organic layers were washed with 2 N aqueous HCl (100 ml), brine, dried over sodium sulfate, and concentrated in vacuo. The residue was purified by column chromatography on silica gel (hexane/ethyl acetate=4:1 as eluent) to afford the titled compound as a yellow solid. (2.5 g , 73%).

¹H-NMR (CDCl₃) δ:10.47 (br.s, 1H), 9.04 (d, J=9.0 Hz, 1H), 8.48 (d, J=2.0 Hz, 1H), 7.94 (dd, J=2.0, 9.0 Hz, 1H), 7.26-7.48 (m, 5H), 3.86 (s, 2H) ppm.

### B. N-(2-amino-4-cyanophenyl)-2-phenylacetamide

A mixture of N-(4-cyano-2-nitrophenyl)-2-phenylacetamide (2.52 g, 8.95 mmol) and 10% Pd/C (100 mg) in methanol (200 ml) was stirred at room temperature under H₂ atmosphere (˜1 atm) for 6 hr. The reaction mixture was filtered through Celite pad and the resulting Pd/C on the celite pad was washed with methanol. The filtrates were concentrated in vacuo. The residue was purified by column chromatography on silica gel (hexane/ethyl acetate=1/8 as eluent) to afford the titled compound as a yellow solid (2.19 g, 97%).

¹H-NMR (CDCl₃) δ:7.34-7.44 (m, 6H), 7.12 (br.s, 1H), 7.00-7.09 (m, 2H), 3.80 (s, 2H), 3.68 (br.s, 2H) ppm.

### C. 2-benzyl-1H-benzimidazole-5-carbonitrile

A mixture of N-(2-amino-4-cyanophenyl)-2-phenylacetamide (2.19 g, 8.71 mmol) and p-toluenesulfonic acid monohydrate (1.49 g, 8.7 mmol) in toluene (250 ml) was refluxed for 5 hr. To the mixture was added 2 N aqueous NaOH (200 mL). The mixture was extracted with ethyl acetate (200 ml×2). The combined organic layers were washed with brine, dried over sodium sulfate, and concentrated in vacuo. The residue was purified by column chromatography on silica gel (hexane/ethyl acetate=1:2 as eluent) to afford the titled compound as a white solid. (981 mg, 48%).

¹H-NMR (CDCl₃) δ:7.26-7.49 (m, 8H), 4.30 (s, 2H) ppm. MS (ESI) 234.09 (M+H)⁺, 232.05 (M−H)⁻,

### D. (2-benzyl-1H-benzimidazol-5-yl)methylamine

A mixture of from 2-benzyl-1H-benzimidazole-5-carbonitrile (881 mg, 3.77 mmol), 25% ammonia solution(4 ml) and Raney-Ni in methanol (40 ml) was stirred under H₂ atmosphere (˜1 atm) for 5 hr. The reaction mixture was filtered through Celite pad and the resulting Pd/C on the Celite pad was washed with methanol. The filtrates were concentrated in vacuo to afford the titled compound as a yellow amorphous (903 mg, 99%).

¹H-NMR (CDCl₃) δ:7.09-7.43 (m, 9H), 4.21 (s, 2H), 3.86 (s, 2H) ppm.

### E. N-[(2-benzyl-1H-benzimidazol-5-yl)methyl]-4-hydroxybenzamide

A mixture of (2-benzyl-1H-benzimidazol-5-yl)methylamine (903 mg, 3.77 mmol), WSC (864 mg, 4.5 mmol), HOBt (560 mg, 4.1 mmol) and 4-Hydroxybenzoic acid (624 mg, 4.5 mmol) in dichloromethane (200 ml) was stirred at room temperature overnight. To the mixture was added water and the mixture was extracted with dichloromethane (100 ml×2). The combined organic layers were washed with brine, dried over sodium sulfate, and concentrated in vacuo. The residue was purified by column chromatography on silica gel (dichloromethane: methanol=20: 1 as eluent) to afford the title compound as a white solid. (409 mg, 29%).

¹H-NMR (CD₃OD) δ:7.12-7.14 (m, 2H), 7.43-7.47 (m, 2H), 7.19-7.30 (m, 6H), 6.81 (d, J=8.9 Hz, 2H9, 4.63 (s, 2H), 4.59 (s, 1H), 4.20 (s, 2H) ppm. IR (KBr)νₘₐₓ:3330, 2491, 1585, 1421, 1359, 1244, 1193, 1145, 1083, 846 cm⁻¹. MS(ESI) 358.0 (M+H)⁺, 356.0 (M−H)⁻

### Example 2

### 4-hydroxy-N-{[1-(2-phenylethyl)-1H-benzimidazol-6-yl]methyl}benzamide

### A. 4-nitro-3-[(2-phenylethyl)amino]benzonitrile

A mixture of 3-chloro-4-nitrobenzonitrile (3 g, 16.4 mmol, Chem. Pharm. Bull., 1992, 2399-2404), 2-phenylethanamine (2.5 ml, 19.7 mmol) and potassium carbonate (3.4 g, 24.6 mmol) in ethanol (200 ml) was refluxed for 5 hr. To the mixture was added 2 N aqueous NaOH (100 ml) and the whole was extracted with ethyl acetate (100 ml×2). The combined organic layers were washed with brine, dried over sodium sulfate, and concentrated in vacuo. The residue was purified by column chromatography on silica gel (hexane/ethyl acetate=1/8 as eluent) to afford the titled compound as a yellow solid (936 mg, 21%).

¹H-NMR (CDCl₃) δ:8.49 (d, J=2.0 Hz, 1H), 8.43 (br.s, 1H), 7.57 (dd, J=2.2, 8.8 Hz, 1H), 7.24-7.39 (m, 5H), 6.89 (d, J=9.0 Hz, 1H), 3.58-3.65 (m, 2H), 3.04 (d, J=7.1 Hz, 2H) ppm.

### B. 4-amino-3-[(2-phenylethyl)amino]benzonitrile

This compound was obtained from 4-nitro-3-[(2-phenylethyl)amino]benzonitrile (936 mg, 3.50 mmol) according to a similar manner to that of Example 1-B as a brown solid. (506 mg, 61%).

¹H-NMR (CDCl₃) δ:7.22-7.42 (m, 5H), 6.99-7.02 (m, 1H), 6.86 (s, 1H), 6.62-6.66 (m, 1H), 3.71 (br.s, 2H), 3.36 (t, J=7.0 Hz, 2H), 3.25 (br.s, 1H), 2.97 (t, J=7.0 Hz, 2H) ppm.

### C. 1-(2-phenylethyl)-1H-benzimidazole-6-carbonitrile

A mixture of 4-amino-3-[(2-phenylethyl)amino]benzonitrile (506 mg, 2.1 mmol) in formic acid (50 ml) was refluxed for 1 hr. To the mixture was added 2 N aqueous NaOH (100 mL). The mixture was extracted with ethyl acetate (100 ml×2). The combined organic layers were washed with brine, dried over sodium sulfate, and concentrated in vacuo to afford the titled compound as white solid (332 mg, 63%).

MS (ESI) 248.10 (M+H)⁺

### D. [1-(2-phenylethyl)-1H-benzimidazol-6-yl]methylamine

This compound was obtained from 1-(2-phenylethyl)-1H-benzimidazole-6-carbonitrile (332 mg, 1.63 mmol) according to a similar manner to that of Example 1-D as a white solid (330 mg, 99%).

MS (ESI) 252.11 (M+H)⁺

### E. 4-hydroxy-N-{[1-(2-phenylethyl)-1H-benzimidazol-6-yl]methyl]benzamide

This compound was obtained from [1-(2-phenylethyl)-1H-benzimidazol-6-yl]methylamine (330 mg, 1.31 mmol) according to a similar manner to that of Example 1-E as a white solid (98 mg, 19%).

¹H-NMR (DMSO-d₆ ) δ:(s, 1H), 7.98 (s, 1H), 7.78 (t, J=3.05 Hz, 2H), 7.54-7.56 (m, 2H), 7.14-7.24 (m, 7H), 6.79-6.81 (m, 2H), 4.57 (d, J=3.05 Hz, 2H), 4.44 (t, J=3.7 Hz, 2H), 3.09 (t, J=3.7 Hz, 2H) ppm. IR (KBr)νₘₐₓ: 1604, 1544, 1282, 1253, 1224, 1176, 1029, 852 cm⁻¹. MS (ESI) 372.10 (M+H)⁺, 369.95 (M−H)⁻

### Example 3

### 4-hydroxy-N-({2-[hydroxy(phenyl)methyl]-1H-benzimidazol-5-yl}methyl)benzamide

### A. 2-benzoyl-1H-benzimidazole-5-carbonitrile

A mixture of 2-benzyl-1H-benzimidazole-5-carbonitrile (Example 1-C , 326 mg, 1.39 mmol) and CrO3 (1.4 g, 13.9 mmol) in acetic acid (50 ml) was stirred at room temperature for 1 day. To the mixture was added water (50 ml) and 2 N aqueous NaOH. The mixture was extracted with ethyl acetate. (50 ml×2). The combined organic layers were washed with brine, dried over sodium sulfate, and concentrated in vacuo. The residue was purified by column chromatography on silica gel (hexane/ethyl acetate=4:1 as eluent) to afford the titled compound as a white solid. (165 g, 48%)

¹H-NMR (CDCl₃) δ:10.66 (br.s, 1H), 8.69-8.73 (m, 2H), 8.34 (s, 1H), 7.97-8.07 (m, 4H), 7.57-7.74 (m, 5H) ppm

### B. 2-(2-phenyl-1,3-dioxolan-2-yl)-1H-benzimidazole-5-carbonitrile

A mixture of 2-benzoyl-1H-benzimidazole-5-carbonitrile (165 mg, 0.66 mmol), ethylene glycol (0.1 ml, 1.33 mmol) and p-toluenesulfonic acid (114 mg, 0.66 mmol) in toluene (60 ml) was refluxed for 5 hr. To the mixture was added water (50 ml) and the mixture was extracted with ethyl acetate. (50 ml×2). The combined organic layers were washed with brine, dried over sodium sulfate, and concentrated in vacuo. The residue was purified by column chromatography on silica gel (hexane/ethyl acetate=1:2 as eluent) to afford the titled compound as a white solid. (162 g, 83%)

¹H-NMR (CDCl₃) δ:8.10 (s, 1H), 7.65-7.68 (m, 2H), 7.49-7.52 (m, 2H), 7.37-7.47 (m, 3H), 4.19-4.24 (m, 4H) ppm

### C. [2-(2-phenyl-1,3-dioxolan-2-yl)-1H-benzimidazol-5-yl]methylamine

This compound was obtained from 2-(2-phenyl-1,3-dioxolan-2-yl)-1H-benzimidazole-5-carbonitrile (162 mg, 0.556 mmol) according to a similar manner to that of Example 1-D as a white amorphous (164 mg, 99%).

¹H-NMR (CDCl₃) δ:7.64-7.75 (m, 2H), 7.28-7.40 (m, 4H), 7.16-7.23 (m,2H), 4.19 (s, 4H), 3.94 (s, 2H) ppm

### D. 4-hydroxy-N-{[2-(2-phenyl-1,3-dioxolan-2-yl)-1H-benzimidazol-5-yl]methyl}benzamide

This compound was obtained from [2-(2-phenyl-1,3-dioxolan-2-yl)-1H-benzimidazol-5-yl]methylamine (164 mg, 0.55 mmol) according to a similar manner to that of Example 1-E as a white solid (76 mg, 33%).

MS (ESI) 415.9 (M+H)⁺, 413.9 (M−H)⁻

### E. N-[(2-benzoyl-1H-benzimidazol-5-yl)methyl]-4-hydroxybenzamide

A mixture of 4-hydroxy-N-{[2-(2-phenyl-1,3-dioxolan-2-yl)-1H-benzimidazol-5-yl]methyl}benzamide (76 mg, 0.18 mmol) in 37% hydrochloric acid (20 ml) was stirred at 50° C. for 12 days. To the mixture was added saturated aqueous NaHCO3 and the mixture was extracted with ethylacetate(50 ml×2). The combined organic layers were washed with brine, dried over sodium sulfate, and concentrated in vacuo. The residue was purified by column chromatography on silica gel (dichloromethane/methanol=30:1 as eluent) to afford the titled compound as a white solid. (42 g, 63%)

¹H-NMR (DMSO-d₆) δ:8.88 (br.s, 1H), 8.56 (d, J=8.7 Hz, 2H), 7.58-7.80 (m, 6H), 7.35 (d, J=8.9 Hz, 1 h), 6.80 (d, J=8.8 Hz, 2H), 4.59 (d, J=5.9 Hz, 2H) ppm

### F. 4-hydroxy-N-({2-[hydroxy(phenyl)methyl]-1H-benzimidazol-5-yl}methyl)benzamide

A mixture of N-[(2-benzoyl-1H-benzimidazol-5-yl)methyl]-4-hydroxybenzamide (23 mg, 0.06 mmol) and sodium borohydride (5 mg, 0.12 mmol) in methanol (15 ml) was stirred at room temperature for 20 min. To the mixture was added water (10 ml) and the mixture was extracted with ethylacetate (30 ml×2). The combined organic layers were washed with brine, dried over sodium sulfate, and concentrated in vacuo. The residue was washed with dichloromethane to afford the titled compound as a white solid. (6 g, 26%)

¹H-NMR (DMSO-d₆) δ:12.28 (br.s, 1H), 9.96 (s, 1H), 8.78 (br.s, 1H), 7.74 (d, J=8.6 Hz, 2H), 7.32-7.34 (m, 3H), 7.24-7.29 (m, 4H), 7.09 (br.s, 1H), 6.79 (d, J=8.8 Hz, 2H), 6.49 (d, J=4.3 Hz, 1H), 5.88 (d, J=3.8 Hz, 1H), 4.49 (d, J=5.7 Hz, 2H) ppm MS (ESI) 374.0 (M+H)⁺, 372.0 (M−H)⁻

### Example 4

### N-[(2-benzyl-1,3-benzoxazol-5-yl)methyl]-4-hydroxybenzamide

### A. methyl 2-benzyl-1,3-benzoxazole-5-carboxylate

A mixture of methyl 3-amino-4-hydroxybenzoate (2 g, 11.9 mmol) and phenylacetyl chloride (1.6 ml, 11.9 mmol) in xylene (200 ml) was refluxed for 2 days. To the mixture was added water (100 ml) and the mixture was extracted with ethylacetate (200 ml×2). The combined organic layers were washed with brine, dried over sodium sulfate, and concentrated in vacuo. The residue was purified by column chromatography on silica gel (hexane/ethyl acetate=4:1 as eluent) to afford the titled compound as a white solid. (3.06 g, 95%)

¹H-NMR (CDCl₃) δ:8.38 (d, J=1.7 Hz, 1H), 8.05 (dd, J=1.7 Hz, 8.7 Hz, 1H), 7.26-7.50 (m, 6H), 4.26 (2H, s), 3.94 (s, 3H)

### B. (2-benzyl-1,3-benzoxazol-5-yl)methanol

To a solution of methyl 2-benzyl-1,3-benzoxazole-5-carboxylate (1.06 g, 3.96 mmol) in tetrahydrofurane (40 ml) was added diisobutylalminum hydride (1.01 M solution in toluene, 5.9 ml, 5.9 mmol) at 0° C. under nitrogen atmosphere. The mixture was stirred at 0° C. for 2 hr. To the mixture was added water (50 ml) and the mixture was extracted with ethyl acetate. (50 ml×2). The combined organic layers were washed with brine, dried over sodium sulfate, and concentrated in vacuo to afford the titled compound as a pale yellow solid. (950 mg, 99%)

¹H-NMR (CDCl₃) δ:7.65 (s, 1H), 7.25-7.43 (m, 7H), 4.75 (s, 2H), 4.26 (s, 2H), 3.93 (s, 1H) ppm

### C. (2-benzyl-1,3-benzoxazol-5-yl)methyl methanesulfonate

A mixture of (2-benzyl-1,3-benzoxazol-5-yl)methanol (950 mg, 3.96 mmol), methanesulfonyl chloride (0.3 ml, 4.3 mmol) and triethylamine (1.1 ml, 7.9 mmol) in dichloromethane (40 ml) was stirred at room temperature under nitrogen atmosphere for 1 hr. To the mixture was added 2 N aqueous NaOH and the mixture was extracted with dichloromethane (50 ml×2). The combined organic layers were washed with brine, dried over sodium sulfate, and concentrated in vacuo to afford the titled compound as a yellow oil (1.02 g, 81%).

MS (ESI) 318.0 (M+H)⁺, 316.0 (M−H)⁻

### D. 5-(azidomethyl)-2-benzyl-1,3-benzoxazole

A mixture from (2-benzyl-1,3-benzoxazol-5-yl)methyl methanesulfonate (1.02 g, 3.21 mmol) and sodium azide (521 mg, 8.02 mmol) in N,N-dimethylformamide (40 ml) was stirred at 130° C. for 2.5 hr. To the mixture was added water (50 ml) and the mixture was extracted with ethylacetate (50 ml×2). The combined organic layers were washed with brine, dried over sodium sulfate, and concentrated in vacuo. The residue was purified by column chromatography on silica gel (hexane/ethyl acetate=8:1) to afford the titled compound as colorless oil (412 mg, 48%).

¹H-NMR (CDCl₃) δ:7.64 (s, 1H), 7.24-7.50 (m, 7H), 4.42 (s, 2H), 4.27 (s, 2H) ppm

### E. (2-benzyl-1,3-benzoxazol-5-yl)methylamine

A mixture of 5-(azidomethyl)-2-benzyl-1,3-benzoxazole (412 mg, 1.56 mmol) and 10% Pd/C (100 mg) in methanol (30 ml) was stirred at room temperature under H₂ atmosphere (˜1 atm) for 3.5 hr. The reaction mixture was filtered through Celite pad and the resulting Pd/C on the celite pad was washed with methanol. The filtrates were concentrated in vacuo to afford the title compound as brown amorphous (282 mg, 75%).

¹H-NMR (CDCl₃) δ:7.61 (s, 1H), 7.24-7.42 (m, 7H), 4.26 (s, 2H), 3.94 (s, 2H) ppm

### F. N-[(2-benzyl-1,3-benzoxazol-5-yl)methyl]-4-hydroxybenzamide

This compound was obtained from (2-benzyl-1,3-benzoxazol-5-yl)methylamine (282 mg, 1.18 mmol) according to a similar manner to that of Example 1-E as a white amorphous (225 mg, 53%).

¹H-NMR (DMSO-d₆) δ:8.83 (t, J=5.1 Hz, 1H), 7.76 (d, J=8.6 Hz, 2H), 7.58 (d, J=7.7 Hz, 2H), 7.27-7.36 (m, 6H), 6.79 (d, J=8.5 Hz, 2H), 4.52 (d, J=5.8 Hz, 2H), 4.32 (s, 2H) ppm IR (KBr)μₘₐₓ:3340, 1629, 1589, 1280, 1242 cm⁻¹. MS(ESI) 359.0 (M+H)⁺

### Example 5

### N-[(2-benzyl-2H-indazol-6-yl)methyl]-4-hydroxybenzamide

### A. methyl 2-benzyl-2H-indazole-6-carboxylate

To a mixture of NaH (540 mg, 13.6 mmol) in N,N-dimethylformamide (20 ml) was added a solution of methyl 1H-indazole-6-carboxylate (2 g, 11.3 mmol, J. Med. Chem., 2000, 41-58) in N,N-dimethylformamide (10 ml) dropwise at room temperature under nitrogen atmosphere. And the resulting mixture was refluxed for 1.5 hr. To the mixture was added benzyl bromide (2 ml, 17.0 mmol) and the mixture was stirred at room temperature for 1.5 hr. To the mixture was added water (50 ml) and the mixture was extracted with ethyl acetate. (50 ml×2). The combined organic layers were washed with brine, dried over sodium sulfate, and concentrated in vacuo. The residue was purified by column chromatography on silica gel (hexane/ethyl acetate=8:1/4:1 as eluent) to afford the titled compound as a white amorphous. (1.07 g, 35%)

¹H-NMR (CDCl₃) δ:8.52 (s, 1H), 7.91 (s, 1H), 7.63-7.72 (m, 2H), 7.26-7.40 (m, 5H), 5.63 (s, 2H), 3.95 (s, 3H) ppm

### B. (2-benzyl-2H-indazol-6-yl)methanol

To a mixture of LiAlH4 (230 mg, 6.0 mmol) in tetrahydrofurane (100 ml) was added a solution of methyl 2-benzyl-2H-indazole-6-carboxylate (1.07 g, 4.0 mmol) in tetrahydrofurane (20 ml) dropwise at 0° C. under nitrogen atmosphere. And the mixture was stirred at 0° C. for 1 hr. To the mixture was added water (50 ml) and the mixture was extracted with ethyl acetate. (50 ml×2). The combined organic layers were washed with brine, dried over sodium sulfate, and concentrated in vacuo to afford the titled compound as a brown amorphous. (956 mg, 98%)

¹H-NMR (CDCl₃) δ:7.82 (s, 1H), 7.55-7.62 (m, 2H), 7.22-7.35 (m, 5H), 7.06 (d, J=7.4 Hz, 1H), 5.54 (s, 2H), 4.70 (s, 2H) ppm

### C. (2-benzyl-2H-indazol-6-yl)methyl methanesulfonate

This compound was obtained from (2-benzyl-2H-indazol-6-yl)methanol (956 mg, 4.0 mmol) according to a similar manner to that of Example4-C as a brown amorphous. (1.2 g, 95%)

¹H-NMR (CDCl₃) δ:8.01 (s, 1H), 7.92 (s, 1H), 7.65-7.76 (m, 4H), 7.29-7.38 (m, 2H), 7.10-7.13 (m, 2H), 5.61 (s, 2H), 4.63 (s, 2), 2.91 (s, 3H) ppm

### D. 6-(azidomethyl)-2-benzyl-2H-indazole

This compound was obtained from (2-benzyl-2H-indazol-6-yl)methyl methanesulfonate (1.29 g, 4.0 mmol) according to a similar manner to that of Example4-D as a pale yellow solid. (550 mg, 52%)

¹H-NMR (CDCl₃) δ:7.88 (s, 1H), 7.64 (d, J=8.1 Hz, 2H), 7.25-7.36 (m, 5H), 7.03 (d, J=8.6 Hz, 1H), 5.59 (s, 2H), 4.41 (s, 2H) ppm

### E. (2-benzyl-2H-indazol-6-yl)methylamine

This compound was obtained from 6-(azidomethyl)-2-benzyl-2H-indazole (550 mg, 2.08 mmol) according to a similar manner to that of Example 4-E as a yellow amorphous. (483 mg, 97%)

¹H-NMR (CDCl₃) δ:7.85 (s, 1H), 7.59 (d, J=8.1 Hz, 2H), 7.26-7.35 (m, 5H), 7.05 (d, J=9.9 Hz, 1H), 5.58 (s, 2H), 3.94 (s, 2H), 1.64 (br.s, 2H) ppm

### F. N-[(2-benzyl-2H-indazol-6-yl)methyl]-4-hydroxybenzamide

This compound was obtained from (2-benzyl-2H-indazol-6-yl)methylamine (483 mg, 2.03 mmol) according to a similar manner to that of Example 1-E as a white amorphous (189 mg, 26%).

¹H-NMR (DMSO-d₆) δ:8.79 (t, J=5.8 Hz, 1H), 8.42 (s, 1H), 7.77 (d, J=8.7 Hz, 2H), 7.64 (d, J=8.6 Hz, 1H), 7.43 (s, 1H), 7.25-7.35 (m, 6H), 5.61 (s, 2H), 4.50 (d, J=6.3 Hz, 2H) ppm IR (KBr)νₘₐₓ:3269, 1629, 1608, 1508, 1276, 1240 cm⁻¹. MS (ESI) 358.0 (M+H)⁺, 356.0 (M−H)⁻

### Example 6

### 4-Hydroxy-N-{[4-(2-phenylethyl)quinolin-6-yl]methyl}benzamide

### A. 4-Hydroxy-N-(4-nitrobenzyl)benzamide

A mixture of 4-hydroxybenzoic acid (4.1 g, 30 mmol), 4-nitrobenzylamine hydrochloride (5.7 g, 30 mmol), triethylamine (8.4 mL, 60 mmol), EDCI (6.9 g, 36 mmol) and HOBt (0.9 g, 6.0 mmol) in DMF (100 mL) was stirred at room temperature for 16 h. The mixture was diluted with AcOEt, and the solution was washed with sat. aq. NaHCO₃ and water. The organic layer was separated, dried over MgSO₄, filtered, and concentrated. The residue was purified by crystallization 2-propanol and diisopropyl ether to afford the titled compound (3.8 g, 14 mmol) as a pale yellow solid.

¹H NMR (270 MHz, DMSO-d₆) δ:10.04 (br, 1H), 8.95 (t, J=6.1 Hz, 1H), 8.21 (d, J=8.4 Hz, 2H), 7.88 (d, J=8.6 Hz, 2H), 7.56 (d, J=8.4 Hz, 2H), 6.83 (d, J=8.6 Hz, 2H), 4.56 (d, J=5.9 Hz, 2H) ppm.

### B. N-(4-Aminobenzyl)4-hydroxybenzamide

A mixture of 4-hydroxy-N-(4-nitrobenzyl)benzamide (3.8 g, 14 mmol) and 10% Pd—C (0.7 g) in ethanol was stirred under hydrogen at 1 atm for 2 h. The mixture was filtered through a pad of Celite. The filtrate was concentrated in vacuo to afford the titled compound (1.6 g, 6.6 mmol) as a white solid.

¹H NMR (270 MHz, DMSO-d₆) δ:10.04 (br, 1H), 8.57 (t, J=5.9 Hz, 1H), 7.74 (d, J=8.7 Hz, 2H), 6.96 (d, J=8.4 Hz, 2H), 6.78 (d, J=8.7 Hz, 2H), 6.50 (d, J=8.4 Hz, 2H), 4.93 (br, 2H), 4.26 (d, J=5.9 Hz, 2H) ppm.

### C. 4-Hydroxy-N-{[4-(2-phenylethyl)guinolin-6-yl]methyl}benzamide

To a mixture of N-(4-aminobenzyl)-4-hydroxybenzamide (82 mg, 0.34 mmol) and 5-phenylpent-1-en-3-one (109 mg, 0.68 mmol) (Synlett 1997, 1414-1416.) in ethanol (7 mL), m-nitrobenznesulfonic acid (62 mg, 0.31 mmol), ZnCl₂ (6 mg, 0.044 mmol) and c.HCl (51 μL) were added and the mixture was refluxed for 4 h. The mixture was cooled to room temperature and was diluted with AcOEt. The solution was washed with sat. aq. NaHCO₃ and water. The aqueous layer was dried over MgSO₄, filtered, and concentrated. The residue was purified by crystallization from dichloromethane and methanol to afford the titled compound (5 mg) as a white solid.

¹H NMR (300 MHz, DMSO-d₆) δ:10.02 (br, 1H), 8.98 (t, J=5.5 Hz, 1H) 8.73 (d, J=4.4 Hz, 1H), 8.08 (s, 1H), 7.99 (d, J=8.6 Hz, 1H), 7.83 (d, J=8.8 Hz, 2H), 7.72 (dd, J=1.7, 8.6 Hz, 1H), 7.38 (d, J=4.4 Hz, 1H), 7.30-7.15 (m, 5H), 6.82 (d, J=8.8 Hz, 2H), 4.70 (d, J=6.0 Hz, 2H), 3.32−3.24 (m, 2H), 3.00−2.90 (m, 2H) ppm. MS (ESI); 383 (M+H)⁺, 381 (M−H)⁻ IR (KBr)νₘₐₓ 3331, 1630, 1609, 1504, 1310, 1286, 1259, 1238, 1173 cm⁻¹

### Example 7

### N-{[8-(Benzyloxy)quinolin-2-yl]methyl}-4-hydroxybenzamide

To a solution of 8-(benzyloxy)quinoline-2-carbaldehyde (80 mg, 0.30 mmol) (Tetrahedron 1996, 52, 4659 - 4672.) in ethanol-water (1:1, 1 mL), hydroxylamine hydrochloride (31 mg, 0.45 mmol) and 2N aq. NaOH (0.45 mL) were added at 0° C. and the mixture was stirred at 0° C. for 30 min. The mixture was diluted with water and extracted with dichloromethane. The extract was dried over MgSO₄ and was evaporated. The residue was dissolved with acetic acid (0.9 mL) and water (0.6 mL). To the solution, zinc (98 mg, 1.5 mmol) was added at 0° C. and the mixture was stirred at 0° C. for 1 h. The mixture was diluted with dichloromethane. To the mixture, K₂CO₃ was added and the suspension was filtered. The filtrate was evaporated. The residue was dissolved with DMF. To the solution, 4-(methoxymethoxy)benzoic acid (22 mg, 0.12 mmol) (Tetrahedron Asymm. 1993, 4, 687-694.), EDCI (23 mg, 0.12 mmol) and HOBt (18 mg, 0.12 mmol) were added and the mixture was stirred at room temperature for 16 h. The mixture was diluted with AcOEt and was washed with sat. aq. NaHCO₃ and water. It was dried over MgSO₄ and was evaporated. The residue was dissolved with HCl-MeOH (1 mL) and the solution was stirred at 50° C. for 3 h. The mixture was diluted with AcOEt and was washed with sat. aq. NaHCO_{3.} It was dried over MgSO₄ and was evaporated. N-{[8-(Benzyloxy)quinolin-2-yl]methyl}-4-hydroxybenzamide (4 mg) was afforded by preparative TLC (hexane-AcOEt 1:2) as a pale brown solid.

¹H NMR (300 MHz, DMSO-d₆) δ=10.01 (s, 1H), 8.99 (t, J=5.9 Hz, 1H) 8.29 (d, J=8.4 Hz, 1H), 7.80 (d, J=8.8 Hz, 2H), 7.56-7.26 (m, 9H), 6.81 (d, J=8.6 Hz, 2H), 5.34 (s, 2H), 4.72 (d, J=6.0 Hz, 2H) ppm. MS (ESI); 385 (M+H)+,383 (M−H)⁻ IR (KBr))νₘₐₓ 3057, 1607, 1535, 1495, 1383, 1279, 1261, 1174, 1101 cm⁻¹

### Example 8

### N-[(2-benzyl-1H-indol-5-yl)methyl]-4-hydroxybenzamide

### A. (2-benzyl-1H-indol-5-yl)methylamine

This compound was obtained from (2-benzyl-1H-indol-5-yl)carbonitrile (250 mg, 1.08 mmol, Bioorg.Med.Chem.Lett., 1996 6 1339-1344.) according to a similar manner to that of Example 1-D as a solid (210 mg, 0.89 mmol).

¹H NMR (300 MHz, CDCl₃) δ 7.80 (br, 1H), 7.46 (s, 1H), 7.35-10 (m, 6H), 7.07 (d, J=8 Hz, 1H), 6.30 (s, 1H), 4.13 (s, 2H), 3.96 (br, 2H), ppm.

### B. N-[(2-benzyl-1H-indol-5-yl)methyl]-4-hydroxybenzamide

This compound was obtained from (2-benzyl-1H-indol-5-yl)methylamine (200 mg, 1.08 mmol) according to a similar manner to that of Example 1-E as amorphous (210 mg, 0.59 mmol).

¹H NMR (300 MHz, DMSO-d₆) δ=10.91 (s, 1H), 9.97 (br, 1H), 8.69 (t, J=6 Hz, 1H), 7.75 (d, J=9 Hz, 2H), 7.34−7.16 (m, 7H), 6.98 (dd, J=8, 2 Hz, 1H), 6.78 (d, J=9 Hz, 2H), 6.10 (s, 1H), 4.46 (d, 2H), 4.03 (s, 2H) ppm

### Example 9

### 4-hydroxy-N-{[1-(2-phenylethyl)-1H-indazol-6-yl]methyl}benzamide

### A. methyl 1-(2-phenylethyl)-1H-indazole-6-carboxylate

This compound was obtained from methyl 1H-indazole-6-carboxylate (2 g,11.35 mmol) according to a similar manner to that of example 5-A as a pale yellow amorphous (1.73 g, 54%).

¹H-NMR (CDCl₃) δ:8.05 (s, 1H), 7.97 (s, 1H), 7.71-7.78 (m, 2H), 7.10-7.25 (m, 5H), 4.65 (t, J=7.4 Hz, 2H), 3.95 (s, 3H), 3.23 (t, J=7.4 Hz, 2H) ppm

### B. [1-(2-phenylethyl)-1H-indazol-6-yl]methanol

This compound was obtained from methyl 1-(2-phenylethyl)-1H-indazole-6-carboxylate (1.73 g, 6.17 mmol)) according to a similar manner to that of example 5-B as a pale yellow amorphous (1.5 g, 96%).

¹H-NMR (CDCl₃) δ:7.90 (s, 1H), 7.65 (d, J=8.3 Hz, 1H), 7.14-7.25 (m, 4H), 7.04-7.10 (m, 3H), 4.75 (d, J=4.8 Hz, 2H), 4.55 (t, J=7.4 Hz, 2H), 3.17 (t, J=7.4 Hz, 2H) ppm

### C. [1-(2-phenylethyl)-1H-indazol-6-yl]methyl methanesulfonate

This compound was obtained from [1-(2-phenylethyl)-1H-indazol-6-yl]methanol (1.5 g , 5.94 mmol) according to a similar manner to that of example 4-C as a pale yellow oil (1.53 g, 78%).

¹H-NMR (CDCl₃) δ:7.99 (s, 1H), 7.67-7.74 (m, 1H), 7.04-7.67 (m, 7H), 4.56-4.70 (m, 4H), 3.23 (s, 3H), 2.87 (s, 2H) ppm

### D. 6-(azidomethyl)-1-(2-phenylethyl)-1H-indazole

This compound was obtained from [1-(2-phenylethyl)-1H-indazol-6-yl]methyl methanesulfonate (1.53 g ,4.63 mmol) according to a similar manner to that of example 4-D as a pale yellow oil (1.13 g, 92%).

¹H-NMR (CDCl₃) δ:8.01 (s, 1H), 7.70 (d, J=8.3 Hz, 1H), 7.00-7.25 (m, 7H), 4.60 (t, J=7.2 Hz, 2H), 4.39 (s, 2H), 3.21 (t, J=7.2 Hz, 2H) ppm

### E. {[1-(2-phenylethyl)-1H-indazol-6-yl]methyl}amine

This compound was obtained from 6-(azidomethyl)-1-(2-phenylethyl)-1H-indazole (1.13 g ,4.29 mmol) according to a similar manner to that of example 4-E as a pale yellow oil (1.01 g, 94%).

¹H-NMR (CDCl₃) δ:7.97-7.98 (m, 1H), 7.63-7.66 (m, 1H), 7.01-7.26 (m, 7H), 4.56-4.61 (m, 2H), 3.93 (s, 2H), 3.20(t, J=7.3 Hz, 2H) ppm

### F. 4-hydroxy-N-{[1-(2-phenylethyl)-1H-indazol-6-yl]methyl}benzamide

This compound was obtained {[1-(2-phenylethyl)-1H-indazol-6-yl]methyl}amine (1.01 g, 4.29 mmol) according to a similar manner to that of example1-E as a white amorphous (514 mg, 32%).

¹H-NMR (DMSO-d₆) δ:8.81 (s, 1H), 8.00 (s, 1H), 7.79 (d, J=8.7 Hz, 2H), 7.67 (d, J=8.2 Hz, 1H), 7.48 (s, 1H), 7.18-7.20 (m, 5H), 7.08 (d, J=8.2 Hz, 1H), 6.82 (d, J=8.7 Hz, 2H), 4.54-4.60 (m, 4H), 3.12 (t, J=7.1 Hz, 2H) ppm IR (KBr)νₘₐₓ:3269, 1629, 1608, 1508, 1276, 1240 cm⁻¹. ES⁺:372.13 (M+1) ES⁻:370.11 (M−1)

### Example 10

### N-{[4-(Benzylamino)quinazolin-6-yl]methyl}-4-hydroxybenzamide

### 4-(Benzylamino)quinazoline-6-carbonitrile

A mixture of 4-chloroquinazoline-6-carbonitrile (0.14 g, 0.73 mmol, WO93/03030), benzylamine (94 mg, 0.88 mmol) and triethylamine (0.11 mL, 0.80 mmol) in CH₂Cl₂ was stirred at room temperature for 16 h. The mixture was treated with sat. aq. NaHCO₃ and was extracted with CH₂Cl₂. The extract was dried over MgSO₄ and was evaporated. The titled compound (0.12 g) was afforded by prep.TLC (hexane-AcOEt 1:3).

¹H NMR (DMSO-d₆) δ:9.22-9.13 (m, 1H), 8.93 (d, J=1.3 Hz, 1H) 8.57 (s, 1H), 8.09 (dd, J=1.8, 8.6 Hz, 2H), 7.81 (d, J=8.6 Hz, 2H), 4.80 (d, J=4.9 Hz, 2H) ppm.

### N-{[4-(Benzylamino)quinazolin-6-yl]methyl}-4-hydroxybenzamide

A mixture of 4-(benzylamino)quinazoline-6-carbonitrile (13 mg, 0.050 mmol), catalytic Raney-Ni and 25% aq. NH₃ (50 μL) in MeOH was hydrogenated at 1 atm for 30 min. The mixture was filtered by celite and the filtrate was evaporated. A mixture of the crude amine, 4-hydroxybenzoic acid (7.0 mg, 0.050 mmol) and HOBt.H₂O (9.0 mg, 0.060 mmol) in DMF (1.0 mL), EDCI (12 mg, 0.060 mmol) was added and the mixture was stirred at room temperature for 16 h. The mixture was diluted with AcOEt and was washed with sat. aq. NaHCO₃ and water. It was dried over MgSO₄ and was evaporated. The titled compound (12 mg) was afforded by prep.TLC (CH₂Cl₂—MeOH 10:1) as a whtie solid.

¹H NMR (DMSO-d₆) δ:9.99 (br, 1H), 8.86 (t, J=6.1 Hz, 2H), 8.41 (s, 1H), 7.81-7.64 (m, 4H), 7.38−7.20 (m, 5H), 6.81 (d, J=8.8 Hz, 2H) , 4.79 (d, J=5.7 Hz, 2H), 4.58 (d, J=5.9 Hz, 2H) ppm. MS (ESI): (M+H)⁺ (385), (M−H)⁻ (383) IR (KBr)νₘₐₓ:1506, 1296, 1246 cm⁻¹

### Example 11

### 4-hydroxy-N-{[2-methyl-1-(2-phenylethyl)-1H-benzimidazol-6-yl]methyl}benzamide

This compound was obtained according to a similar procedure to that of example 2 as a white solid.

¹H-NMR (DMSO-d₆ ) δ:9.64 (s, 1H), 8.35 (s, 1H), 7.82 (s, 1H), 7.51 (d, J=6.1 Hz, 1H), 7.36 (s, 1H), 7.19-7.22 (m, 4H), 6.94-6.97 (m, 2H), 6.82 (d, J=8.7 Hz, 2H), 4.67 (d, J=5.5 Hz, 2H), 4.33 (t, J=5.6 Hz, 2H), 3.22 (t, J=5.6 Hz, 2H), 2.14 (s, 3H) ppm. IR (KBr)νₘₐₓ:1508, 1411, 1255, 1172, 1105, 846 cm⁻¹. ES⁺:386.18 (M+1) ES⁻:384.15 (M−1)

### Example 12

### N-{[4-(Benzyloxy)quinolin-6-yl]methyl}-4-hydroxybenzamide)

### Benzyl 4-(benzyloxy)quinoline-6-carboxylate

A mixture of ethyl 4-chloroquinoline-6-carboxylate (0.62 g, 2.6 mmol, J. Med. Chem. 1994, 37, 2106-2111.) and sodium benzyloxide (1.0M solution in benzyl alcohol, 2.9 mL, 2.9 mmol) was stirred at 150° C. for 16 h. The mixture was quenched with sat. aq. NH₄Cl and was extracted with CH₂Cl₂. The extract was dried over MgSO₄ and was evaporated. The titled compound (0.38 g) was afforded by silica-gel column chromatography (hexane-AcOEt 1:1).

¹H NMR (CDCl₃) δ:9.10-9.05 (m, 1H), 8.81 (d, J=5.3 Hz, 1H), 8.32 (dd, J=2.0, 8.9 Hz, 1H), 8.07 (d, J=8.7 Hz, 1H) , 7.53-7.32 (m, 11H), 5.44 (s, 2H), 5.35 (s, 2H) ppm.

### [4-(Benzyloxy)quinolin-6-yl]methanol

To a solution of benzyl 4-(benzyloxy)quinoline-6-carboxylate (0.37 g, 1.0 mmol) in THF, DIBAL-H (0.95 M in hexane, 3.2 mL, 3.0 mmol) was added at 0° C. and the mixture was stirred at 0° C. for 1 h. The mixture was quenched with water and was extracted with CH₂Cl₂. The extract was dried over MgSO₄ and was evaporated. The titled compound (0.19 g) was afforded by silica-gel column chromatography (hexane-AcOEt 2:3).

¹H NMR (CDCl₃) δ:8.71 (d, J=5.3 Hz, 1H), 8.25-8.21 (m, 1H), 8.03 (d, J=8.4 Hz, 1H), 7.71 (dd, J=2.0, 8.6 Hz, 1H), 7.53−7.34 (m, 5H), 6.80 (d, J=5.3 Hz, 1H), 5.29 (s, 2H), 4.88 (s, 2H) ppm.

### 6-(Azidomethyl)-4-(benzyloxy)quinoline

To a mixture of [4-(benzyloxy)quinolin-6-yl]methanol (80 mg, 0.30 mmol) and triethylamine (83 μL, 0.60 mmol) in CH₂Cl₂, methanesulfonylchloride (26 μL, 0.33 mmol) was added at 0° C. and the mixture was stirred at 0° C. for 2 h. The mixture was quenched with water and was extracted with CH₂Cl₂. The extract was dried over MgSO₄ and was evaporated. A mixture of the crude mixture and NaN₃ (85 mg, 1.3 mmol) in DMF (1.3 mL) was stirred at 50° C. for 2 h. The mixture was diluted with AcOEt and was washed with water. It was dried over over MgSO₄ and was evaporated. The titled compound (8.0 mg) was afforded by silica-gel column chromatography (hexane-AcOEt 2:1).

¹H NMR (CDCl₃) δ:8.76 (d, J=5.3 Hz, 1H), 8.19 (d, J=2.0 Hz, 1H), 8.07 (d, J=8.6 Hz, 1H), 7.67 (dd, J=2.0, 8.6 Hz, 1H) , 7.54−7.39 (m, 5H), 6.84 (d, J=5.1 Hz, 1H) , 5.32 (s, 2H), 4.53 (s, 2H) ppm.

### N-{[4-(Benzyloxy)quinolin-6-yl]methyl}-4-hydroxybenzamide

A mixture of 6-(azidomethyl)-4-(benzyloxy)quinoline (8.0 mg, 28 μmol), triphenylphosphine (11 mg, 42 μmol) and water (28 μL) in THF (0.30 mL) was stirred at room temperature for 24 h. The mixture was diluted with CH₂Cl₂ and was extracted with 2N aq. HCl. The acidic extract was alkalized with 2N aq. NaOH and it was extracted with CH₂Cl₂. The extract was dried over MgSO₄ and was evaporated. To a mixture of the crude, 4-(acetyloxy)benzoic acid (3.6 mg, 20 μmol) and HOBt.H₂O (3.0 mg, 20 μmol) in DMF (0.50 mL), EDCI (3.8 mg, 20 μmol) was added and the mixture was stirred at room temperature for 16 h. To the mixture, 2N aq. NaOH (1 mL) and MeOH (1 mL) were added and the mixture was stirred at room temperature for 2 h. The mixture was neutrized with 2N aq. HCl. It was extracted with AcOEt and the extract was washed with sat. aq. NaHCO₃ and water. It was dried over MgSO₄ and was evaporated. The titled compound (1.6 mg) was afforded by prep.TLC (CH₂Cl₂—MeOH 10: 1) as a whtie solid.

¹H NMR (DMSO-d₆) δ:10.07 (br, 1H), 8.97-9.90 (m, 1H), 8.70 (d, J=5.1 Hz, 1H), 8.10−8.07 (m, 1H), 7.92 (d, J=8.8 Hz, 1H) , 7.80−7.68 (m, 3H), 7.54−7.48 (m, 2H), 7.43−7.33 (m, 3H), 7.11 (d, J=5.3 Hz, 1H) , 6.81 (d, J=8.6 Hz, 2H) , 5.38 (s, 2H), 4.62 (d, J=5.9 Hz, 2H) ppm.

### Example 13

### 4-hydroxy-N-{[2-oxo-3-(2-phenylethyl)-2,3-dihydro-1H-benzimidazol-5-yl]methyl}benzamide

### A. 2-oxo-3-(2-phenylethyl)-2,3-dihydro-1H-benzimidazole-5-carbonitrile

A mixture of 3-chloro-4-nitrobenzonitrile (620 mg, 2.4 mmol, Chem. Pharm. Bull, (1992) 2399-2404), 1,1-carbonyldiimidazole (778 mg, 4.8 mmol) in tetrahydrofurane (30 ml) was stirred at room temperature overnight. To the mixture was added water (30 ml) and the whole was extracted with ethyl acetate (100 ml×2). The combined organic layers were washed with brine, dried over sodium sulfate, and concentrated in vacuo. The residue was purified by column chromatography on silica gel (hexane/ethyl acetate=1/1 as eluent) to afford the titled compound as a white solid (415 mg, 65%).

¹H-NMR (CDCl₃) δ:10.96 (s, 1H), 7.17-7.30 (m, 2H), 7.05 (d, J=8.1 Hz, 1H), 6.89 (d, J=1.3 Hz, 1), 4.07 (t, J=7.0 Hz), 3.02 (t, J=7.0 Hz, 2H) ppm.

### B. 6-(aminomethyl)-1-(2-phenylethyl)-1,3-dihydro-2H-benzimidazol-2-one

This compound was obtained from 2-oxo-3-(2-phenylethyl)-2,3-dihydro-1H-benzimidazole-5-carbonitrile (415 mg, 1.57 mmol) according to a similar manner to that of example1-D as a yellow solid (442 mg, 99%).

¹H-NMR (CDCl₃) δ:7.15-7.28 (m, 5H), 6.96-7.03 (m, 2H), 6.72-6.80 (m, 1H), 4.11 (t, J=7.2 Hz, 2H), 3.77-3.83 (m, 2H), 3.05 (t, J=7.2 Hz, 2H) ppm.

### C. 4-hydroxy-N-{[2-oxo-3-(2-phenylethyl)-2,3-dihydro-1H-benzimidazol-5-yl]methyl}benzamide

This compound was obtained from 6-(aminomethyl)-1-(2-phenylethyl)-1,3-dihydro-2H-benzimidazol-2-one (442 mg, 1.57 mmol) according to a similar manner to that of example1-E as a white solid (98 mg, 43%).

¹H-NMR (DMSO-d₆ ) δ:10.75 (s, 1H), 9.96 (s, 1H), 8.71 (t, J=5.8 Hz, 1H), 7.78 (d, J=8.7 Hz, 2H), 7.17-7.27 (m, 5H), 7.06 (s, 1H), 6.87-6.94 (m, 2H), 6.80 (d, J=8.8 Hz, 2H), 4.44 (d, J=5.8 Hz, 2H), 3.96 (t, J=7.1 Hz, 2H), 2.91 (t, J=7.1 Hz, 2H) ppm. IR (KBr)νₘₐₓ:1546, 1363, 1172, 1107.985 cm⁻¹. ES⁺:388.23 (M+1) ES⁻:386.21 (M−1)

### Example 14

### 4-hydroxy-N-{[3-(2-phenylethyl)-1H-indazol-5-yl]methyl}benzamide]

### A. 4-fluoro-3-(1-hydroxy-3-phenylpropyl)benzonitrile

To a solution of 4-fluoro-3-formylbenzonitrile (688 mg, 4.6 mmol Tetrahedron Lett., 1992, 7499-7502) in tetrahydrofurane (15 ml) was added phenethyl magnesium bromide (15 ml, 0.3 M solution in tetrahydrofurane ) dropwise at −78° C. under nitrogen atmosphere. The mixture was stirred at room temperature overnight. To the mixture were added water (50 ml) and the mixture was extracted with ethyl acetate (50 ml×2). The combined organic layers were washed with brine, dried over sodium sulfate, and concentrated in vacuo. The residue was purified by column chromatography on silica gel (hexane/ethyl acetate=8:1/4:1 as eluent) to afford the titled compound as a colorless oil. (463 mg, 39%)

¹H-NMR (CDCl₃ ) δ:7.87 (dd, J=2.2 Hz, 6.8 Hz, 1H), 7.53-7.59 (m, 1H), 7.08-7.32 (m, 6H), 5.05 (q, J=5.5 Hz, 1H), 2.72-2.84 (m, 2H), 2.02-2.12 (m, 2H) ppm.

### B. 4-fluoro-3-(3-phenylpropanoyl)benzonitrile

A mixture of 4-fluoro-3-(1-hydroxy-3-phenylpropyl)benzonitrile (463 mg, 1.81 mmol) and 3-pyridinesulfonic acid (865 mg, 5.44 mmol) and triethylamine (1.3 ml, 9 mmol) in dimethylsulfoxide (18 ml) was stirred under nitrogrn atmosphere at room temperature 2 hr. To the mixture were added water (50 ml) and the mixture was extracted with ethyl acetate (50 ml×2). The combined organic layers were washed with brine, dried over sodium sulfate, and concentrated in vacuo. The residue was purified by column chromatography on silica gel (hexane/ethyl acetate=10/1 as eluent) to afford the titled compound as a white solid. (388 mg, 85%)

¹H-NMR (CDCl₃ ) δ:8.17-8.20 (m, 1H), 7.77-7.92 (m, 1H), 7.21-7.32 (m, 6H), 3.28-3.47 (m, 2H), 3.06 (t, J=7.6 Hz, 2H) ppm.

### C. 3-(2-phenylethyl)-1H-indazole-5-carbonitrile

A mixture of 4-fluoro-3-(3-phenylpropanoyl)benzonitrile (388 mg, 1.53 mmol) and hydrazine (0.2 ml, 6.12 mmol) in dimethylsulfoxide (10 ml) was stirred at 80° C. for 2 hr. To the mixture were added water (50 ml) and the mixture was extracted with ethyl acetate (50 ml×2). The combined organic layers were washed with brine, dried over sodium sulfate, and concentrated in vacuo. The residue was purified by column chromatography on silica gel (hexane/ethyl acetate=4:1/1:1 as eluent) to afford the titled compound as colorless oil. (327 mg, 86%)

¹H-NMR (CDCl₃ ) δ:7.85 (s, 1H), 7.47-7.56 (m, 2H), 7.17-7.33 (m, 5H), 3.32 (t, J=7.0 Hz, 2H), 3.13 (t, J=7.0 Hz, 2H) ppm.

### D. }[3-(2-phenylethyl)-1H-indazol-5-yl]methyl}amine

This compound was obtained from 3-(2-phenylethyl)-1H-indazole-5-carbonitrile (227 mg, 0.92 mmol) according to a similar manner to that of example1-D as a yellow oil (220 mg, 95%).

### E. 4-hydroxy-N-{[3-(2-phenylethyl)-1H-indazol-5-yl]methyl}benzamide

This compound was obtained from {[3-(2-phenylethyl)-1H-indazol-5-yl]methyl}amine 220 mg, 0.87 mmol) according to a similar manner to that of example1-E as a white solid (85 mg, 26%).

¹H-NMR (DMSO-d₆) δ:12.60 (s, 1H), 8.76 (t, J=6.1 Hz, 1H), 7.76 (d, J=8.6 Hz, 2H), 7.63 (s, 1H), 7.14-7.42 (m, 7H), 6.80 (d, J=8.6 Hz, 2H), 4.52 (d, J=6.1 Hz, 2H), 3.01-3.19 (m, 4H) ppm IR (KBr)νₘₐₓ:3280, 1616, 1575, 1508, 1271, 1174, 1107 cm⁻¹. ES⁺:372.25 (M+1) ES⁻:370.18 (M−1)

### Example 15

### 4-Hydroxy-N-{[3-(2-phenylethyl)imidazo[1,5-a]pyridin-6-yl]methyl}benzamide

### A. Methyl 6-{[(3-phenylpropanoyl)amino]methyl}nicotinate

To a mixture of methyl 6-(aminomethyl)nicotinate (261 mg, 1.57 mmol, prepared according to C. Ingrid C et.al., J. Med. Chem., 2002, 45, 5005) in pyridine (5 ml) was added 3-phenylpropanoyl chloride (291 mg, 1.73 mmol) at 0° C. and stirred for 0.5 hr. To the reaction mixture was added sat. NaHCO₃ aq. (15 ml) and was extracted with dichloromethane (20 ml×3). The combined organic layers were washed with brine (20 ml), dried over sodium sulfate, and concentrated in vacuo. The residue was purified by column chromatography on silica gel (hexane/acetone=4:1 as eluent) to afford the titled compound as a white solid (227 mg, 49%).

¹H-NMR (DMSO-d₆) δ:9.00−8.99 (m, 1H), 8.55−8.53 (m, 1H), 8.19−8.16 (m, 1H), 7.32−7.15 (m, 6H), 4.40 (d, J=5.9 Hz, 2H), 3.88 (s 3H), 2.89−2.83 (m, 2H), 2.54−2.51 (m, 2H) ppm. MS (ESI): 299.10 (M+H)⁺

### B. Methyl 3-(2-phenylethyl)imidazo[1,5-a]pyridin-6-carboxylate

To a solution of methyl 6-{[(3-phenylpropanoyl)amino]methyl}nicotinate (227 mg, 0.76 mmol) in 1,2-dichloroethane (30 ml) was added phosphorus oxychloride (0.35 ml, 3.81 mmol) and refluxed for 1 hr. The mixture was concentrated in vacuo, and the pH of the combined mixture was adjusted to 8.0 with sat. NaHCO₃ aq. The mixture was extracted with dichloromethane (20 ml×3), dried over sodium sulfate, and concentrated in vacuo. The residue was was purified by column chromatography on silica gel (hexane/ethyl acetate=5:1 as eluent) to afford the titled compound as yellow oil (156 mg, 73%).

¹H-NMR (CDCl₃) δ:8.33−8.32 (m, 1H), 7.41−7.11 (m, 8H), 3.91 (s, 3H), 3.35−3.29 (m, 2H), 3.25−3.19 (m, 2H) ppm.

### C. [3-(2-Phenylethyl)imidazo[1,5-a]pyridin-6-yl}methanol

To a solution of methyl 3-(2-phenylethyl)imidazo[1,5-a]pyridin-6-carboxylate (156 mg, 0.56 mmol) in tetrahydrofuran (20 ml) was added LiAlH₄ (32 mg, 0.83 mmol) and the reaction mixture was stirred at 0° C. for 1 hr. To the mixture was added sat. Na₂SO₄ aq. (0.4 ml) and was stirred at room temperature for 5 min. The combined mixture was filtered with celite and the filtrate was concentrated in vacuo. The residue was purified by column chromatography on silica gel (dichloromethane/methanol=20:1 as eluent) to afford the titled compound as yellow oil (105 mg, 75%).

¹H-NMR (CDCl₃) δ:7.45−7.44 (m, 1H), 7.35−7.12 (m, 7H), 6.63−6.59 (m, 1H), 4.54−4.53 (m, 2H), 3.22−3.08 (m, 4H) ppm. MS (ESI): 253.15 (M+H)⁺

### E. 6-(Azidomethyl)-3-(2-phenylethyl)imidazo[1,5-a]pyridine

To a solution of [3-(2-phenylethyl)imidazo[1,5-a]pyridin-6-yl]methanol (31 mg, 0.12 mmol) in THF (1.5 ml) was added a 1.6 M solution of n-butyllithium in hexane (85 μl, 0.14 mmol) at −78° C. and the mixture was stirred for 15 min. To the reaction mixture was added methanesulfonyl chloride (16 mg, 0.14 mmol) in THF (1.5 ml) and the mixture was gradually warmed up to 0° C. for 3 hr. Furthermore, to the mixture was added a 1.6 M solution of n-butyllithium in hexane (0.17 ml, 0.27 mmol) and methanesulfonyl chloride (31 mg, 0.27 mmol) at −78° C. and the mixture was gradually warmed up to 0° C. for 2 hr. To the reaction mixture was added sat. NaHCO₃ aq (10 ml). The mixture was extracted with dichloromethane (15 ml×3), dried over sodium sulfate, and concentrated in vacuo to afford 56 mg of material. Without further purification, to the crude material in DMF (3 ml) was added sodium azide (16 mg, 0.25 mmol) and stirred at 70° C. for 1 day. To the reaction mixture was poured sat. NaHCO₃ aq. (5 ml), and was extracted with dichloromethane (15 ml×3). The combined organic layers were dried over sodium sulfate, and concentrated in vacuo. The residue was purified by column chromatography on silica gel (hexane/ethyl acetate=1:2 as eluent) to afford the titled compound as yellow oil (8.5 mg, 25%).

¹H-NMR (CDCl₃) δ:7.43−7.40 (m, 3H), 7.31−7.18 (m, 5H), 6.59−6.55 (m, 1H), 4.19 (s, 2H), 3.30−3.17 (m, 4H) ppm. MS (ESI): 278.19 (M+H)⁺

### F. }[3-(2-Phenylethyl)imidazo[1,5-a]pyridin-6-yl]methyl}amine

A mixture of 6-(azidomethyl)-3-(2-phenylethyl)imidazo[1,5-a]pyridine (17 mg, 0.06 mmol)and 10% palladium on carbon (2.2 mg) in methanol (2 ml) was stirred at room temperature under hydrogen for 15 hr. The reaction mixture was filtered on celite and the filtrate was evaporated to afford the titled compound as yellow oil (13 mg, 85%).

¹H-NMR (CDCl₃) δ:7.44−7.17 (m, 8H), 6.60−6.57 (m, 1H), 3.71 (s, 2H), 3.30−3.15 (m, 4H) ppm. [NH₂ proton was not observed.] MS (EST): 252.14 (M+H)⁺

### G. 4-(Methoxymethoxy)-N-{[3-(2-phenylethyl)imidazo[1,5-a]pyridin-6-yl]methyl}benzamide

To a mixture of {[3-(2-phenylethyl)imidazo[1,5-a]pyridin-6-yl]methyl}amine (11 mg, 0.04 mmol), 4-(methoxymethoxy)benzoic acid (8.2 mg, 0.05 mmol) and triethylamine (8.3 mg, 0.08 mmol) in DMF (2 ml) was added WSC (12 mg, 0.06 mmol) and HOBt (10 mg, 0.06 mmol) at room temperature and the mixture was stirred for 1 day. To the reaction mixture was poured sat. NaHCO₃ aq. (10 ml), and was extracted with dichloromethane (15 ml×3). The combined organic layers were dried over sodium sulfate, and concentrated in vacuo. The residue was purified by column chromatography on silica gel (dichloromethane/methanol=20:1 as eluent) to afford the titled compound as colorless oil (5.9 mg, 34%).

¹H-NMR (CDCl₃) δ:7.76−7.73 (m, 2H), 7.41−7.35 (m, 3H), 7.25−7.07 (m, 7H), 6.64−6.61 (m, 1H), 6.25 (brs, 1H), 5.22 (s, 2H), 4.44 (d, J=5.9 Hz, 2H), 3.48 (s, 3H), 3.27−3.14 (m, 4H) ppm. MS (ESI): 416.33 (M+H)⁺, 414.29 (M−H)⁻

### H. 4-Hydroxy-N-{[3-(2-phenylethyl)imidazo[1,5-a]pyridin-6-yl]methyl}benzamide

A solution of 4-(methoxymethoxy)-N-{[3-(2-phenylethyl)imidazo[1,5-a]pyridin-6-yl]methyl}benzamide (5.9 mg, 0.01 mmol) in 10% hydrogen chloride in methanol (1.5 ml) was stirred at 50° C. for 45 min. The reaction mixture was concentrated in vacuo. The residue was dissolved to dichloromethane (10 ml) and the pH of the mixture was adjusted to 8.0 with sat. NaHCO₃ aq. The mixture was extracted with dichloromethane (15 ml×3), dried over sodium sulfate, and concentrated in vacuo. The residue was was purified by column chromatography on silica gel (dichloromethane/methanol=20:1 as eluent) to afford the titled compound as a white solid (4.7 mg, 93%).

¹H-NMR (DMSO-d₆) δ:9.99 (brs, 1H), 8.71 (m, 1H), 8.04 (s, 1H), 7.76 (d, J=8.4 Hz, 2H), 7.47 (d, J=9.0 Hz, 1H), 7.28−7.18 (m, 6H), 6.80 (d, J=8.6 Hz, 2H), 6.72 (d, J=9.9 Hz, 1H), 4.38−4.36 (m, 2H), 3.25−3.03 (m, 4H) ppm. MS (ESI): 372.26 (M+H)⁺, 370.23 (M−H)⁻

### Example 16

### N-{[3-(benzyloxy)-1,2-benzisoxazol-5-yl]methyl}-4-hydroxybenzamide

### B. methyl 3-(benzyloxy)-1,2-benzisoxazole-5-carboxylate

A mixture of methyl 3-hydroxy-1,2-benzisoxazole-5-carboxylate (342 mg, 1.77 mmol, Chem. Ber., 1967, 954-960), benzylalcohol (0.25 ml, 2.12 mmol), triphenylphosphine (557 mg, 2.12 mmol) and Diethy azodicarboxylate (40% in toluene, 1.15 g, 2.65 mmol) in tetrahydrofurane was stirred at room temperature overnight. The solvent was removed and the residue was purified by column chromatography on silica gel (hexane/ethyl acetate=1:1 as eluent) to afford the titled compound as a yellow solid. (403 mg, 80%).

¹H-NMR (CDCl₃) δ:8.40 (s, 1H), 8.24 (dd, J=1.7 Hz, 8.9 Hz, 1H), 7.41-7.55 (m, 6H), 5.48 (s, 2H), 3.93 (s, 3H) ppm.

### B. [3-(benzyloxy)-1,2-benzisoxazol-5-yl]methanol

To a mixture of methyl 3-(benzyloxy)-1,2-benzisoxazole-5-carboxylate (100 mg, 0.35 mmol) in tetrahydrofurane (10 ml) and ethanol (10 ml) was added sodium borohydride (14.6 mg, 0.38 mmol). Then to the mixture was added LiCl (16.4 mg, 0.38 mmol). The mixture was stirred at room temperature for 4 days. To the mixture was added water (50 ml) and the whole was extracted with ethyl acetate(100 ml×2). The combined organic layers were washed with brine, dried over sodiun sulfate, and concentrated in vacuo. The residue was purified by column chromatography on silica gel (hexane/ethyl acetate=1:1 as eluent) to afford the titled compound as a white solid. (20 mg, 22%).

¹H-NMR (CDCl₃) δ:7.64-7.65 (m, 1H), 7.49-7.53 (m, 3H), 7.38-7.45 (m, 4H), 5.45 (s, 2H), 4.76 (s, 2H) ppm.

### C. [3-(benzyloxy)-1,2-benzisoxazol-5-yl]methyl methanesulfonate

This compound was obtained from [3-(benzyloxy)-1,2-benzisoxazol-5-yl]methanol (230 mg, 0.90 mmol) according to a similar manner to that of example4-C as a colorless oil (247 mg, 82%).

¹H-NMR (CDCl₃) δ:7.74 (s, 1H), 7.41-7.62 (m, 7H), 5.47 (s, 2H), 5.32 (s, 2H), 2.95 (s, 3H) ppm.

### D. 5-(azidomethyl)-3-(benzyloxy)-1,2-benzisoxazole

This compound was obtained from [3-(benzyloxy)-1,2-benzisoxazol-5-yl]methyl methanesulfonate (247 mg, 0.74 mmol) according to a similar manner to that of example4-D as a colorless oil (203 mg, 97%).

¹H-NMR (CDCl₃) δ:7.41-7.62 (m, 8H), 5.47 (s, 2H), 4.43 (s, 2H) ppm.

### E. {[3-(benzyloxy)-1,2-benzisoxazol-5-yl]methyl}amine

This compound was obtained from 5-(azidomethyl)-3-(benzyloxy)-1,2-benzisoxazole (100 mg, 0.36 mmol) according to a similar manner to that of example4-E as a white solid (33 mg, 25%).

### F. N-{[3-(benzyloxy)-1,2-benzisoxazol-5-yl]methyl}-4-hydroxybenzamide

This compound was obtained from {[3-(benzyloxy)-1,2-benzisoxazol-5-yl]methyl}amine(33 mg, 0.09 mmol) according to a similar manner to that of example 1-E as a white solid (11 mg, 32%).

¹H-NMR (CDCl₃): 7.60-7.66 (m, 3H), 7.47-7.53 (m, 3H), 7.36-7.42 (m, 4H), 6.82 (d, J=8.7 Hz, 2H), 6.48 (s, 1H), 5.43 (s, 2H), 4.69 (d, J=5.9 Hz, 2H) ppm. IR (KBr)νₘₐₓ, 3265, 1637, 1541, 1500, 1272, 1238 cm⁻¹. ES⁺:375.14 (M+1) ES⁻:373.04 (M−1)

### Example 17

### N-{[2-(2-fluorobenzyl)-1H-benziniidazol-6-yl]methyl}-4-hydroxybenzamide

This compound was obtained according to a similar procedure to that of example 1 as a white solid.

¹H-NMR (DMSO-d₆ ) δ:8.73 (s, 1H), 7.74 (d, J=8.5 Hz, 4H), 7.27-7.38 (m, 4H), 7.07-7.21 (m, 3H), 6.70 (s, 2H), 4.50 (d, J=5.8 Hz, 2H), 4.18 (s, 2H) ppm. ES⁺:376.14 (M+1) ES⁻:374.12 (M−1)

### Example 18

### N-[(2-benzyl-5,6,7,8-tetrahydroimidazo[1,2,-a]pyridin-7-yl)methyl]-4-hydroxybenzamide

### A. (7-methylimidazo[1,2-a]pyridin-2-yl)(phenyl)methanone

Bromine (1.0 ml, 19.7 mmol) was dropwise added to a solution of benzil (2.92 g, 19.7 mmol) in ether (20 ml). The mixture was stirred overnight and quenched with saturated Na2S2O3aq. (100 ml) The whole was extracted with ether (100 ml×2). The combined organic layers were washed with brine(20 ml), dried over MgSO4, and concentrated in vacuum. The residue was dissolved with ethanol (50 ml) and 2-amino-4-methylpyridine (2.14 g, 19.6 mmol) was added. The resulting mixture was stirred for 1 hour at reflux temperature. After concenration, the residue was purified on SiO2, eluting with hexane-ethyl acetate (1:1) to afford to afford the titled compound. (0.91 g, 20%)

¹H-NMR (CDCl3) δ:8.34−8.28 (m, 2H), 8.15 (s, 1H), 8.04 (d, J=7.0 Hz, 1H), 7.63−7.43 (m, 4H), 6.72 (dd, J=7.0 Hz, 1.7 Hz, 1H), 2.42 (s, 3H) ppm

### B. [3-chloro-7-(chloromethyl)imidazo[1,2-a]pyridin-2-yl](phenyl)methanone

A mixture of (7-methylimidazo[1,2-a]pyridin-2-yl)(phenyl)methanone (0.79 g, 3.34 mmol), NCS (0.89 g, 6.69 mmol) and TFA (0.6 ml) in ethyl acetate (20 ml) was stirred overnight and quenched with saturated NaHCO3 aq.(30 ml) The whole was extracted with ethyl acetate (30 ml×2). The combined organic layers were washed with brine(30 ml), dried over MgSO4, and concentrated in vacuum. The residue was purified on SiO2, eluting with hexane-ethyl acetate (4:1) to afford to afford the titled compound. (0.45 g, 44%)

¹H-NMR (CDCl3) δ:8.34−8.26 (m, 2H), 8.24−8.18 (m, 1H), 7.70−7.43 (m, 5H), 7.12−7.06 (m, 1H), 4.65 (s, 2H) ppm

### C. [7-(azidomethyl)-3-chloroimidazo[1,2-a]pridin-2-yl](phenyl)methanone

A mixture of [3-chloro-7-(chloromethyl)imidazo[1,2-a]pyridin-2-yl](phenyl)methanone (0.45 g, 1.47 mmol), sodium azide (192 mg, 2.95 mmol) and 15-crown-5 (324 mg, 1.47 mmol) in THF (12 ml) was stirred overnight at 70° C. and quenched with water (20 ml). The whole was extracted with ethyl acetate (20 ml×2). The combined organic layers were washed with brine(20 ml), dried over MgSO4, and concentrated in vacuum. The residue was purified on SiO2, eluting with hexane-ethyl acetate (5:1) to afford to afford the titled compound. (332 mg, 73%)

¹H-NMR (CDCl₃) δ:8.32−8.19 (m, 3H), 7.67-7.48 (m, 4H), 7.00 (dd, J=1.7 Hz, 7.2 Hz, 1H), 4.47 (s, 2H) ppm.

### D. N-[(2-benzoylimidazo[1,2-a]pydridin-7-yl)methyl]-4-hydroxybenzamide

A mixture of [7-(azidomethyl)-3-chloroimidazo[1,2-a]pyridin-2-yl](phenyl)methanone (150 mg) and 10%-Pd/C (50 mg) in methanol (10 ml) was stirred for 6 hours under hydrogen (1.5 kg/cm2) at room temperature. After filtration by celite pad, the filtrate was concentrated in vacuum. The residue was dissolved with DMF (5 ml). To the mixture were added 4-methoxymethoxybenzoic acid (0.2 g), WSC (0.3 g), and HOBt (0.2 g). The mixture was stirred overnight and quenched with water (15 ml). The whole was extracted with ethyl acetate (20 ml×2). The combined organic layers were washed with brine(20 ml), dried over MgSO4, and concentrated in vacuum. The residue was purified on SiO2, eluting with hexane-ethyl acetate (2:1) to afford N-[(2-benzoylimidazo[1,2-a]pyridin-7-yl)methyl]-4-(methoxymethoxy)benzamide (43 mg). A mixture of N-[(2-benzoylimidazo[1,2-a]pyridin-7-yl)methyl]-4-(methoxymethoxy)benzamide (43 mg) and 10%HCl/MeOH (3 ml) in methanol (3 ml) was stirred for 2 hours at 50° C. and quenched with saturated NaHCO3aq. (10 ml) The whole was extracted with ethyl acetate (10 ml×2). The combined organic layers were washed with brine, dried over MgSO₄, and concentrated in vacuum. The residue was purified by preparative TLC to afford the titled compound. (5.2 mg)

¹H-NMR (DMSO) δ:10.02 (s, 1H), 8.90 (t, J=5.9 Hz, 1H), 8.62−8.53 (m, 2H), 8.33−8.26 (m, 2H), 7.85−7.46 (m, 6H), 7.05−6.96 (m, 1H), 6.83 (d, J=8.6 Hz, 2J=5.9 Hz, 1H) ppm ES⁺:372.11 (M+1) ES⁻:370.08 (M−1)

### E. 4-hydroxy-N-(}2-[hydroxy(phenyl)methyl]imidazo [1,2-a]pyridin-7-yl}methyl)benzamide

A mixture of N-[(2-benzoylimidazo[1,2-a]pyridin-7-yl)methyl]-4-hydroxybenzamide (4.2 mg, mmol) and 10%-Pd/C (5 mg) in methanol (3 ml) was stirred for 6 hours under hydrogen (4 kg/cm2) at room temperature. After filtration by celite pad, the filtrate was concentrated in vacuum. The residue was purified by preparative TLC to afford the titled compound. (3.2 mg)

¹H-NMR (DMSO) δ:9.87 (s, 1H), 8.86−8.78 (m, 1H), 8.41 (d, J=7.0 Hz, 1H), 7.79−7.67 (m, 3H), 7.43−7.18 (m, 5H), 6.83−6.75 (m, 3H), 5.87−5.72 (m, 3H), 4.43 (d, J=5.3 Hz, 2H) ppm ES⁺:374.10 (M+1) ES⁻:372.07 (M−1)

### F. N-[(2-benzyl-5,6,7,8-tetrahydroimidazo[1,2-a]pyridin-7-yl)methyl]-4-hydroxybenzamide

A mixture of 4-hydroxy-N-({2-[hydroxy(phenyl)methyl]imidazo[1,2-a]pyridin-7-yl}methyl)benzamide (2.2 mg, mmol) and Pd(OH)₂/C (5 mg) in methanol (3 ml) was stirred for 6 hours under hydrogen (4 kg/cm2) at room temperature. After filtration by celite pad, the filtrate was concentrated in vacuum. The residue was purified by preparative TLC to afford the titled compound. (1.12 mg)

¹H-NMR (DMSO) δ:9.96 (bs, 1H), 8.36−8.29 (m, 1H), 7.72 (d, J=8.6 Hz, 2H), 7.30−7.20 (m, 5H), 6.79 (d, J=8.6 Hz, 2H), 6.62 (s, 1H), 4.03−3.90 (m, 1H), 3.80−3.68 (m, 3H), 3.50−3.20 (m, 2H), 2.83−2.73 (m, 1H), 2.42−1.94 (m, 3H), 1.64−1.50 (m, 1H) ppm ES⁺:362.10 (M+1) ES⁻:360.09 (M−1)

### Example 19

### 4-hydroxy-N-{[2-methyl-1-(2-phenylethyl)-1H-benzimidazol-6-yl]methyl]benzamide

This compound was obtained according to a similar procedure to that of example 2 as a white solid.

¹H-NMR (DMSO-d₆ ) δ:9.98 (s, 1H), 8.78 (s, 1H), 8.00 (s, 1H), 7.89 (d, J=8.8 Hz, 2H), 7.47-7.57 (m, 2H), 7.05-7.18 (m, 4H), 6.80 (d, J=8.8 Hz, 2H), 4.56 (d, J=6.0 Hz, 2H), 4.45 (t, J=6.7 Hz, 2H), 3.13 (t, J=6.7 Hz, 2H) ppm.

### Example 20

### N-[(2-benzyl-1H-indol-5-yl)methyl]-3-fluoro-4-hydroxybenzamide Sodium Salt

This compound was obtained according to a similar procedure to that of example 8 as amorphous.

¹H-NMR (DMSO-d₆) δ:10.09 (br, 1H), 8.01 (br, 1H), 7.32−7.16 (m, 11H), 6.95 (dd, J=8, 2 Hz, 1H), 6.10 (br, 2H), 4.40 (d, J=6 Hz, 2H) ppm.

### Example 21

### 4-hydroxy-N-{[1-(2-phenylethyl)-1H-imidazo[4,5-b]pyridin-6-yl]methyl}benzamide

### A. 3H-imidazo[4,5-b]pyridine-6-carbonitrile

This compound was obtained 5,6-diaminonicotinonitrile (750 mg, 5.59 mmol Ger. Offen. 1987, 22) according to a similar manner to that of example2-C as a white amorphous (174 mg, 21%).

¹H-NMR (DMSO-d₆) δ:8.78 (s, 1H), 8.70 (s, 1H), 8.63 (s, 1H) ppm.

### B. 1-(2-phenylethyl)-1H-imidazo[4,5-b]pyridine-6-carbonitrile

This compound was obtained from 3H-imidazo[4,5-b]pyridine-6-carbonitrile (442 mg, 1.57 mmol) according to a similar manner to that of example5-A as a white solid (53 mg, 18%).

¹H-NMR (DMSO-d₆) δ:8.78 (d, J=2.0 Hz, 1), 8.62-8.64 (m, 2H), 7.14-7.24 (m, 5H), 4.59 (t, J=7.1 Hz, 2H), 3.14 (t, J=7.1 Hz, 2H) ppm.

### C. }[1-(2-phenylethyl)-1H-imidazo[4,5-b]pyridin-6-yl]methyl}amine

This compound was obtained from 1-(2-phenylethyl)-1H-imidazo[4,5-b]pyridine-6-carbonitrile (53 mg, 0.21 mmol) according to a similar manner to that of example1-D as a white solid (53 mg, 99%).

¹H-NMR (CDCl₃ ) δ:8.48 (d, J=1.8 Hz, 1H), 7.83 (s, 1H), 7.55 (d, J=1.8 Hz, 1H), 7.23-7.26 (m, 5H), 6.97-7.01 (m, 1H), 4.42 (t, J=6.9 Hz, 2H), 4.01 (s, 2H), 3.13 (t. J=6.9 Hz, 2H) ppm.

### D. 4-hydroxy-N-{[1-(2-phenylethyl)-1H-imidazo[4,5-b]pyridin-6-yl]methyl}benzamide

This compound was obtained from {[1-(2-phenylethyl)-1H-imidazo[4,5-b]pyridin-6-yl]methyl }amine (53 mg, 0.21 mmol) according to a similar manner to that of example1-E as a white solid (19 mg, 24%).

¹H-NMR DMSO-d₆) δ:8.82 (t, J=5.9 Hz, 1H), 8.38 (d, J=1.8 Hz, 1H), 8.25 (s, 1H), 7.94 (d, J=1.8 Hz, 1H), 7.77 (d, J=8.7 Hz, 2H), 7.11-7.24 (m, 5H), 6.81 (d, J=8.7 Hz, 2H), 4.57 (d, J=5.6 Hz, 2H), 4.50 (t, J=7.2 Hz, 2H), 3.10 (t, J=7.2 Hz, 2H) ppm ES⁺:373.13 (M+1) ES⁻:371.13 (M−1)

1. A compound of the formula (I): 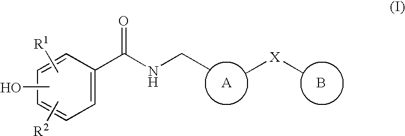
wherein
R¹ and R² independently represent a hydrogen atom, a halogen atom, an alkyl group having from 1 to 6 carbon atoms, an alkoxy group having from 1 to 6 carbon atoms, a cyano group, an alkanoyl group having from 1 to 6 carbon atoms, a haloalkyl group having from 1 to 6 carbon atoms, or a haloalkoxy group having from 1 to 6 carbon atoms;
X represents a covalent bond, an alkylene group having from 1 to 3 carbon atoms, an alkylene group having from 1 to 3 carbon atoms substituted by a hydroxy group or an oxo group; a methyleneoxy group, an ethyleneoxy group, a methyleneoxymethylene group, an oxymethylene group, an ethyleneoxy group, oxy, imino, iminomethylene, iminoethylene, methyleneimino or ethyleneimino, said imino groups are unsubstituted or are substituted by an alkyl group having from 1 to 6 carbon atoms;
A represents a bicyclic, aromatic, saturated or partially unsaturated heterocyclic or carbocyclic group having from 8 to 12 ring atoms;
said heterocyclic group contains either from 1 to 4 nitrogen atoms, or 1 or 2 nitrogen atoms and/or 1 or 2 oxgen or sulfur atoms,
said heterocyclic or carbocyclic group are unsubstituted or are substituted by at least one substituent selected from the group consisting of substituents α;
B represents a phenyl group or a heteroaryl group having from 5 to 6 ring atoms; said phenyl groups and said heteroaryl groups having from 5 to 6 atoms are unsubstituted or are substituted by at least one substituent selected from the group consisting of substituents α;
said substituents a are selected from the group consisting of halogen atoms, alkyl groups having from 1 to 6 carbon atoms, alkoxy groups having from 1 to 6 carbon atoms, cyano groups, alkanoyl groups having from 1 to 6 carbon atoms, haloalkyl groups having from 1 to 6 carbon atoms, oxo groups or haloalkoxy groups having from 1 to 6 carbon atoms;
or a pharmaceutically acceptable ester of such compound;
or a pharmaceutically acceptable salt thereof.

2. A compound according to claim 1 wherein:
R¹ and R² independently represent a hydrogen atom or a fluorine atom.

3. A compound according to claim 1 wherein:
X represents an alkylene group having from 1 to 2 carbon atoms, an alkylene group having from 1 to 2 carbon atoms substituted by a hydroxy group or an oxo group, a methyleneoxy group, an oxymethylene group, iminomethylene or methyleneimino,
said imino groups are unsubstituted or are substituted by an alkyl group having from 1 to 6 carbon atoms.

4. A compound according to claim 1 wherein:
X represents an alkylene group having from 1 to 2 carbon atoms, an oxymethylene group or iminomethylene.

5. A compound according to claim 1 wherein
A represents a bicyclic aromatic heterocyclic group having from 8 to 10 ring atoms, said heterocyclic group contains either from 1 to 3 nitrogen atoms, or 1 nitrogen atom and/or 1 oxygen or atom.

6. A compound according to claim 1 to 5 wherein
A represents a benzimidazole group, a benzoisoxazole group, an indole group, an indazole group, a quinazolin group, an oxo-1H-benzimidazole group, an imidazopyridine group, a tetrahydroimidazopyridine group, or a quinoline group.

7. A compound according to claim 1 wherein
B represents an optionally substituted phenyl group.

8. A compound according to claim 1 wherein
B represents unsubstituted phenyl group or a fluorophenyl group.

9. A compound according to claim 1 selected from:
N-[(2-benzyl-1H-benzimidazol-5-yl)methyl]-4-hydroxybenzamide;
4-hydroxy-N-{[1-(2-phenylethyl)-1H-benzimidazol-6-yl]methyl}benzamide;
N-[(2-benzyl-1H-indol-5-yl)methyl]-4-hydroxybenzamide;
4-hydroxy-N-{[1-(2-phenylethyl)-1H-indazol-6-yl]methyl}benzamide;
N-{[4-(Benzylamino)quinazolin-6-yl]methyl}-4-hydroxybenzamide;
4-hydroxy-N-{[2-methyl-1-(2-phenylethyl)-1H-benzimidazol-6-yl]methyl}benzamide;
N-{[4-(Benzyloxy)quinolin-6-yl]methyl}-4-hydroxybenzamide;
4-hydroxy-N-{[2-oxo-3-(2-phenylethyl)-2,3-dihydro-1H-benzimidazol-5-yl]methyl }benzamide;
4-hydroxy-N-{[3-(2-phenylethyl)-1H-indazol-5-yl]methyl}benzamide];
4-Hydroxy-N-{[3-(2-phenylethyl)imidazo[1,5-a]pyridin-6-yl]methyl}benzamide;
N-{[3-(benzyloxy)-1,2-benzisoxazol-5-yl]methyl}-4-hydroxybenzamide;
N-{[2-(2-fluorobenzyl)-1H-benzimidazol-6-yl]methyl}-4-hydroxybenzamide;
N-[(2-benzyl-5,6,7,8-tetrahydroimidazo[1,2-a]pyridin-7-yl)methyl]-4-hydroxybenzamide;
N-[(2-benzyl-1H-indol-5-yl)methyl]-3-fluoro-4-hydroxybenzamide; and
4-hydroxy-N-{[1-(2-phenylethyl)-1H-imidazo[4,5-b]pyridin-6-yl]methyl}benzamide;
or a pharmaceutically acceptable salt thereof.

10. A pharmaceutical composition, which comprises a compound according to claims 1, or a pharmaceutically acceptable ester of such compound, or a pharmaceutically acceptable salt thereof, and a suitable pharmaceutically acceptable carrier.

11. A pharmaceutical composition for the treatment of disease conditions caused by overactivation of NMDA NR2B receptor, in a mammalian subject, which comprises a therapeutically effective amount of a compound according to claims 1, or a pharmaceutically acceptable ester of such compound, or a pharmaceutically acceptable salt thereof, and a suitable pharmaceutically acceptable carrier.

12. A method for the treatment of disease conditions caused by overactivation of NMDA NR2B receptor, in a mammalian subject, which comprises administering to said subject a therapeutically effective amount of a compound according to claims 1, or a pharmaceutically acceptable ester of such compound, or a pharmaceutically acceptable salt thereof.

13. A method according to claim 12 where the disease condition is selected from stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse, alcohol abuse and Irritable bowel syndrome (IBS).
